SEQUENCE LISTING (4 sequences)

SEQ ID NO: 1 - DNA, 19 bp, synthetic construct
  source 1..19: mol_type = other DNA
caatggtcgtgccaaaaag

SEQ ID NO: 2 - DNA, 21 bp, synthetic construct
  source 1..21: mol_type = other DNA
ttcacatacagcttgggaagc

SEQ ID NO: 3 - DNA, 19 bp, synthetic construct
  source 1..19: mol_type = other DNA
agaccctgttccagccatc

SEQ ID NO: 4 - DNA, 18 bp, synthetic construct
  source 1..18: mol_type = other DNA
tgctagggccgtgatctc

(19) United States
(12) Patent Application Publication
(10) Pub. No.: US 2026/0234549 A1
(43) Pub. Date: Aug. 13, 2026

(54) NON-INVASIVE TUNING OF PROTEIN ALIGNMENT

(71) Applicant: NEW YORK UNIVERSITY IN ABU DHABI CORPORATION, Abu Dhabi (AE)

(72) Inventors: Jeremy C.M. Teo, Abu Dhabi (AE); Jiranuwat Sapudom, Abu Dhabi (AE); Anna Garcia-Sabaté, Singapore (SG); Brian Chesney Quartey, Abu Dhabi (AE)

(21) Appl. No.: 19/154,593

(22) PCT Filed: Feb. 7, 2024
(86) PCT No.: PCT/IB2024/051112

§ 371 Date: Aug. 7, 2025

Related U.S. Application Data
(60) Provisional application No. 63/483,676, filed on Feb. 7, 2023.

(51) Int. Cl. C12N 5/00; C07K 14/78

(52) U.S. Cl. CPC C12N 5/0068; C07K 14/78; C12N 2533/54

(57) ABSTRACT

The present disclosure provides a method for aligning fibrils (e.g., protein fibrils, such as, for example, collagen fibrils) in a three-dimensional matrix. The alignment may be described via coherence index (CI). CI is a range of 0 to 1, where 0 corresponds to randomly oriented fibrils, and 1 corresponds to perfect alignment. The fibrils aligned via a method of the present disclosure may have a CI greater than or equal to 0.1, but less than 1. The resulting matrix may have a variety of uses, including, but not limited to, cell culture, tissue mimetics, cell differentiation, or as a drug screening substrate.

### CROSS REFERENCE TO RELATED APPLICATIONS

This application claims the benefit of U.S. Provisional Application No. 63/483,676, filed Feb. 7, 2023, the disclosure of which is incorporated herein by reference.

### SEQUENCE LISTING

The instant application contains a Sequence Listing, which is submitted in .xml format and is hereby incorporated by reference in its entirety. Said .xml file is named “058636_00682_ST26.xml”, was created on Feb. 5, 2024, and is 4,610 bytes in size.

### BACKGROUND OF THE DISCLOSURE

Alignment of collagen fibrils is a hallmark of fibrotic tissues, which include cancer microenvironment, obesity-associated diseases, chronic inflammation, and scarring. It has been shown that the alignment of collagen fibrils not only provides changes in microarchitectural organization, but also mechanical properties of the tissue. During fibrosis, gradual changes in mechanical properties of collagen can be observed due to the alteration in its microstructure and organization, namely collagen fibril thickening and alignment. The change in the extracellular matrix (ECM) parameters might have a consequence to cells residing within and, thus, alter their functions and phenotypes. The major cells that are involved in the fibrosis are fibroblasts and different subtypes of macrophages. The abundant cells found at fibrotic sites when compared to normal tissues are myofibroblasts. These differentiated fibroblasts are characterized by high expression of alpha-smooth muscle actin (αSMA), excessive matrix component production and a high contractile capability. The differentiation of fibroblasts into myofibroblasts is largely regulated by transforming growth factor beta-1 (TGF-β1), however, mechanical properties of the ECM can also trigger fibroblast differentiation via cell contractility. Recent works have shown that both fibroblasts and macrophages phenotypically change in response to mechanical and physical cues of their immediate surrounding microenvironment. Dynamic study on macrophages-fibroblast interactions in a co-culture model demonstrated that macrophages preferentially migrate towards the matrices actively deformed by fibroblasts and microarchitectural change can be sensed by macrophages found several hundreds of micrometers away. Moreover, the percentage of attracted macrophages decreases with increasing collagen alignment. However, less is known whether the gradual change in matrix organization alters the fate decision and cellular functions of fibroblasts, which will pave the way for understanding the fibrotic progression and development of therapeutic strategies for tissue fibrosis.

Due to the physiological and pathological relevance of collagen alignment, there have been many studies using various methods to mimic this aligned ECM microstructure to serve as biomimetic models of a range of tissues. For example, continuous cyclic stretching of a collagen sheet is used to rearrange collagen fibrils to a high degree of alignment, serving as a substrate for two-dimensional (2D) culture studies. Other methods made use of microfluidic channels with controlled fluid flow allowing for shear forces or magnetic particles enmeshed in collagen hydrogel with an externally applied magnetic field to induce the alignment of collagen fibrils during the collagen fibrillation process. However, the success of achieving aligned collagen hydrogel through the latter approach has an average success rate of only 50% and this is highly dependent on bead size and surface modification. These approaches also rely on the viscosity of collagen solution and, thus, have to be optimized for desired concentration and batch-to-batch variations. Another widely used method has to do with the use of three-dimensional (3D) microfabrication techniques where natural or synthetic bioinks are printed in a shear-induced aligned form. Despite the variety of methods developed for mimicking alignment of collagen fibrils in vitro, these methods are invasive, low-throughput and may require specialized equipment and skill sets. The reconstitution of such matrices may not accurately mimic the in vivo ECM alignment, leaving foreign residuals (e.g., magnetic particles), and generally do not allow for adjusting degree of fibril alignment.

### SUMMARY OF THE DISCLOSURE

The present disclosure provides a method for aligning fibrils (e.g., protein fibrils, such as, for example, collagen fibrils) in a three-dimensional matrix. The resulting matrix may have a variety of uses, including, but not limited to, cell culture, tissue mimetics, cell differentiation, or as a drug screening substrate.

In an aspect, the present disclosure provides a method for aligning fibrils (e.g., protein fibrils, such as, for example, collagen fibrils). The fibrils are at least partially aligned. The alignment may be described via coherence index (CI). CI is a range of 0 to 1, where 0 corresponds to randomly oriented fibrils, and 1 corresponds to perfect alignment. The fibrils aligned via a method of the present disclosure may have a CI greater than or equal to 0.1 but less than 1, greater than or equal to 0.11 but less than 1, greater than or equal to 0.12 but less than 1, greater than or equal to 0.13 but less than 1, greater than or equal to 0.14 but less than 1, greater than or equal to 0.15 but less than 1, greater than or equal to 0.16 but less than 1, greater than or equal to 0.17 but less than 1, greater than or equal to 0.18 but less than 1, greater than or equal to 0.19, or greater than or equal to 0.2 but less than 1 but less than 1.

In an aspect, the present disclosure provides a three-dimensional hydrogel matrix. For example, the hydrogel matrix comprises one or more proteins that self-assemble to form protein fibrils. For example, the protein may be collagen (e.g., type I collagen, type II collagen, type III collagen, type IV collagen, or any combination thereof). The hydrogen matrix may be made by a method of the present disclosure.

In an aspect, the present disclosure provides an assembly 100 upon which substrates 101 may be arranged, as shown in FIGS. 18-21.

The assembly 100 includes a first structure 110 and a second structure 120—for example, a first polygonal structure and a second polygonal structure. The first structure 110 and the second structure 120 are at least partially separated (i.e., divided) by a gap 130, and wherein the first structure 110 and the second structure 120 have a base surface 112, 122 and an oblique surface 111, 121 relative to the base surface 112, 122. The assembly 100 is configured to hold a work piece 101 (e.g., a petri dish or other substrate capable of cell growth or protein self-assembly). The gap 130 allows for improved access to the substrate 101 for the application of heat to the substrate 101. The assembly 100 may comprise a first structure 110 and a second structure 120 being separate elements (as shown in FIG. 18) or a monolithic structure connected by a connecting structure 140 (as shown in FIG. 19). The first structure 110 and the second structure 120 may be solid elements or hollow frame elements (as shown in FIG. 20). The first structure 110 and the second structure 120 may include grooves 113, 123 that are configured to retain the work piece 101 on the substrate surfaces 111, 121.

### BRIEF DESCRIPTION OF THE FIGURES

For a fuller understanding of the nature and objects of the disclosure, reference should be made to the following detailed description taken in conjunction with the accompanying figures.

FIG. 1. Reconstitution and characterization of 3D collagen matrices with adjustable fibril alignment degree. (A) Schematic illustration of the developed approach to reconstitute collagen alignment using 3D-printed inclined surfaces with defined angles. Collagen solution was transferred onto functionalized coverslips and placed onto the inclined surfaces prior to collagen fibrillation under standard cell culture conditions. (B) Representative image of collagen matrices reconstituted on inclined surfaces with angles of 0°, 150 and 30°. Arrows represent the direction of alignment. (C) Representative distribution plot of collagen fibril orientation of collagen matrices reconstituted on inclined surfaces with angles of 0°, 150 and 30°. (D) Quantitative analysis of the coherence index (CI) indicates the degree of collagen fibril alignment. The grey dashed line represents the linear fitting. For the quantification of CI, 4 different positions of each matrix condition were analyzed. Data are shown as box plots: box . . . median with 10th and 90th percentiles, error bars . . . minimum and maximum values. * indicates statistical significance of p<0.05 using the Mann-Whitney test. Experiments were performed in 4 replicates.

FIG. 2. Topological and mechanical characterization of reconstituted 3D collagen matrices. Topological analysis of 3D collagen matrices for (A) mean pore diameter and (B) mean fibril diameter was performed using a custom-made image analysis toolbox. Three different positions of each matrix condition from 4 different samples were analyzed. (C) Bulk matrix elastic modulus was quantified using a non-destructive contactless rheometer from 4 different samples of each matrix condition Data are shown as box plots: box . . . median with 10th and 90th percentiles, error bars . . . minimum and maximum values. * indicates statistical significance of p<0.05 using the Mann-Whitney test.

FIG. 3: Cell orientation and elongation of fibroblasts in reconstituted 3D matrices with aligned and random fibrils. (A) Representative images of fibroblasts cultured onto reconstituted 3D collagen matrices. Arrows represent the direction of alignment. (B) Distribution plot of the orientation of the cell body in dependence on collagen fibril organization. (C) Quantitative analysis of the cell aspect ratio as an indicator of cell elongation in dependence on collagen fibril organization. At least 200 cells were analyzed from 4 different positions and 4 matrices of each matrix condition. Data are shown as box plots: box . . . median with 10th and 90th percentiles, error bars . . . minimum and maximum values. * indicates statistical significance of p<0.05 using the Mann-Whitney test.

FIG. 4: Transcriptome analysis using RNA sequencing of fibroblasts cultured onto matrices with randomly organized and aligned collagen fibrils Functional transcriptome analysis was analyzed using the iDEP web-based tool. (A) Heat map of overall gene expression levels. (B) Principal component analysis of gene expression data. (C) The Venn diagram shows up- and downregulated differentially expressed genes with an FDR cutoff of 0.05 and a minimal fold change of 2. Analysis of biological processes of cells cultivated under (D) 30° vs 0° conditions, (E) 30° vs 0°+EDC conditions and (F) 30° conditions vs myofibroblasts using the generally applicable gene-set enrichment (GAGE) method. RNA sequencing was performed in 3 independent experiments.

FIG. 5. Fibroblast differentiation in matrices with randomly organized and aligned collagen fibrils. Fibroblasts were cultured onto reconstituted matrices for 3 days. Fibroblasts were analyzed regarding their differentiation into myofibroblasts, proliferative capacity, and cytokine secretion. Arrows represent the direction of alignment. (A) Representative images of fibroblasts stained with Hoechst-33421 (nucleus), phalloidin (actin) and αSMA (myofibroblast marker; grey) (scale bar=20 μm). (B) The number of αSMA-positive cells was manually counted from the obtained images. At least 4 random positions per matrix were analyzed. (C) The expression of αSMA was confirmed using RT-qPCR. (D) The number of cells was analyzed by counting cells using flow cytometry. Data were normalized to cell number from 0° conditions. Experiments were performed in 4 replicates. * indicates a significance level of p<0.05 using the Mann-Whitney test. (E) Expression of secreted cytokines analyzed from RNA-Seq data. Data are presented as a heat map. Symbols, o (0° vs 0°+EDC), * (0° vs 30°), § (0° vs myoFB), #(30° vs myoFB), indicate a significance level of p<0.05 using the Mann-Whitney test. (F) Cytokine secretion of CCL2, CXCL8, IL-6, TGF-β1 and VEGF were analyzed using multiplex-bead based ELISA and quantified using flow cytometry. Experiments were performed in 4 replicates. * indicates a significant level of p<0.05 using Mann-Whitney test.

FIG. 6: Matrix remodeling by fibroblasts in matrices with randomly organized and aligned fibrils. Fibroblasts were cultured onto reconstituted matrices for 3 days. Matrices were then decellularized and characterized regarding their topological and mechanical properties. (A) Representative images of decellularized matrices (scale bar=20 μm). Topological parameters, namely, (B) matrix porosity and (C) fibril diameter, were quantified using a custom-built image analysis toolbox. (D) The change in matrix elasticity was calculated by normalizing the obtained data to the matrix elasticity before cell seeding. Data are presented as a dot plot with mean and standard deviation. Experiments were performed in at least 4 replicates. * indicates a significance level of p<0.05 using the Mann-Whitney test. (E) Expression of matrix components analyzed from RNA-Seq data. Data are presented as a heat map (red-high expression; white-low expression). The symbols o (0° vs 0°+EDC), * (0° vs 30°), § (0° vs myoFB), and #(30° vs myoFB) indicate a significance level of p<0.05 using the Mann-Whitney test.

FIG. 7: Inhibition of YAP, contractility and TGF-β1 signaling of fibroblasts in matrices with aligned collagen fibrils. Fibroblasts were cultured on matrix with aligned collagen fibrils (30° condition) in the presence of verteporfin, blebbistatin, Y-27632 and SB-43154 for 3 days. Cells were analyzed regarding (A) αSMA expression and (B) percentage of dead cells. For αSMA expression, data were normalized to fibroblasts. * indicates a significance level of p<0.05 using the Mann-Whitney test. Experiments were performed in 6 replicates. (C) Representative images of the cell orientation of cells cultured on matrix with aligned collagen fibrils (30° condition) with and without treatment with blebbistatin and Y-27632. Cells were stained with Hoechst-33421 (nucleus) and phalloidin (actin) (scale bar=50 μm). The arrow represents the direction of alignment. (D) Proposed mechanotransduction mechanism by which collagen fibril alignment triggers fibroblast differentiation.

FIG. 8: Microstructure of reconstituted matrices. Representative image of collagen matrices reconstituted on inclined surfaces with angles of 7.5°, 22.5° and 0° with postmodification with EDC crosslinker (scale bar=20 μm). Arrows represent the direction of alignment.

FIG. 9: Thickness of collagen matrices reconstituted on inclined surface with an angle of 30°. Representative confocal image shows a cross-sectional view of a 5 mm xz-section of collagen matrices that were reconstituted on inclined surfaces with angles of 30°. Image was gathered using 10× objective (Leica, Wetzlar, Germany). Depth coding profile—color corresponds to the depth from the collagen surface, with blue being closest to the surface and red to the coverslip. In addition, zoomed-in images of four different sections were illustrated, and the collagen thickness was measured (indicated by text and scale). The image demonstrates that the thickness of the collagen reconstituted on the inclined surface minimally changed with an incline of approximately 0.23°.

FIG. 10: Long-range images of collagen matrices with aligned fibrils. Representative images of xy-sections were obtained from collagen matrices reconstituted on an inclined surface with an angle of 30°. Images from two positions were acquired using a 40× oil immersion objective (Leica, Wetzlar, Germany) and analyzed using the OrientationJ plug-in of Fiji. Colors in the images indicate fibril orientation, and the distribution of fibril direction for each position was also plotted. The images demonstrate that collagen alignment can be observed on a long-range scale of the matrix.

FIG. 11: The coherence index of an aligned (ideal) scenario and a simulated collagen fibril arrangement. Different patterns were manually generated, and the coherence index (CI) was quantified for each pattern using the OrientationJ plug-in of Fiji software. The image highlights the difference in CI between a perfectly aligned (ideal) scenario and a simulated scenario of collagen fibril arrangement. It can be observed that, in the collagen fibril scenario, an increase in the number of aligned fibrils leads to a slight change in the CI value, from 0.41 to 0.49.

FIG. 12: The reconstruction of aligned collagen matrices with different collagen concentrations. Representative images were obtained from collagen matrices reconstituted on an inclined surface with an angle of 300 at collagen concentrations of 1 mg/mL and 3 mg/mL. Images were analyzed using the OrientationJ plug-in of Fiji, and colors in the images indicate fibril orientation. The distribution of fibril direction for each position was also plotted. The result demonstrate that our developed method can be used to reconstitute collagen matrices with different types of collagen and concentrations, which enables us to mimic various physiological and pathological conditions.

FIG. 13: The reconstruction of aligned collagen matrices with type I bovine collagen. Representative images were obtained from collagen matrices reconstituted on an inclined surface with an angle of 300 at a concentration of 2 mg/mL with 250 mM phosphate buffer at pH 7.5 and pH 6.5, which is similar to the type I rat tail collagen used in this study. Images were analyzed using the OrientationJ plug-in of Fiji, and colors in the images indicate fibril orientation. The distribution of fibril direction for each position was also plotted. The result demonstrate that our developed method can be used to reconstitute collagen matrices with other sources of type I collagen.

FIG. 14: The reconstruction of aligned collagen matrices with different fibril thicknesses. Representative images were obtained from collagen matrices reconstituted on an inclined surface with an angle of 300 at a collagen concentration of 2 mg/mL 250 mM phosphate buffer at pH 7.5 and pH 6.5. Images were analyzed using the OrientationJ plug-in of Fiji, and colors in the images indicate fibril orientation. The distribution of fibril direction for each position was also plotted. The result demonstrate that our developed method can be used to reconstitute collagen matrices with different types of collagen and concentrations, which enables us to mimic various physiological and pathological conditions.

FIG. 15: Topological and mechanical characterization of reconstituted matrices. (A) Representative images of collagen matrices (Scale bar=20 μm). Arrows indicate the direction of fibril alignment. Matrix topology was quantified using image-based analysis toolboxes. (B) Representative distribution plot of collagen fibril orientation. (C) Quantitative analysis of the coherence index, indicating the degree of collagen fibril alignment. (D) Quantitative analysis of mean pore diameter and (E) mean fibril diameter. For topological analysis, four different positions of each matrix condition from four different samples were analyzed. (F) Bulk matrix elastic modulus was quantified using a non-destructive contactless rheometer from four different samples of each matrix condition. Data are shown as box plots: the box represents the median with the 10th and 90th percentiles, error bars indicate the minimum and maximum values, and a plus sign (+) represents the mean of the data. An asterisk (*) indicates statistical significance (p<0.05) as determined by the Mann-Whitney test.

FIG. 16: Immunophenotyping and cytokine secretion profiling of differentiated macrophages (M0) on reconstituted matrices. (A) A schematic illustration depicts the differentiation of macrophages within 3D collagen matrices. (B) Representative images display the morphology of differentiated macrophages after resting for 72 h on reconstituted matrices. (Scale bar=20 μm). For immunophenotyping, cells were stained with specific antibodies and analyzed using a flow cytometer. (C) Quantification of the geometric mean of fluorescence intensity (gMFI) for HLADR, CD105, CD163, and CD206 in differentiated macrophages immediately after differentiation (0 h) and after resting for 72 h. (D) A co-expression plot of HLADR and CD105, as well as a quantitative analysis of CD206-positive macrophages after 72 h of resting. (E) Cytokine secretion profiles were quantified using multiplex-bead ELISA for macrophages after 72 h of resting. Data are presented as a heatmap, with blue indicating low secretion and red signifying high secretion. An asterisk (*) indicates statistical significance (p<0.05) as determined by the Mann-Whitney test. Experiments were performed in 6 independent replicates.

FIG. 17: Transcriptome analysis of macrophages cultivated in different matrices. Functional transcriptome analysis was conducted using the iDEP web-based tool. (A) A heatmap displaying the overall gene expression levels in macrophages in various matrices. (B) Principal component analysis and (C) correlation matrix of gene expression data. (D) The Venn diagram illustrates the up- and downregulated differentially expressed genes with an FDR cutoff of 0.05 and a minimum fold change of 2. Functional transcriptome analysis of (E) biological activity and (F) curated pathway interaction database of cells cultivated on different matrices (blue represents low expression; red represents high expression). (G) Schematic illustration of KEGG pathway analysis of the PI3K-AKT signaling pathway. The colored box indicates the up- or downregulation of genes involved in the pathways (left box: random EDC vs random; right box: aligned vs random). RNA sequencing was conducted in three independent experiments.

FIG. 18: An assembly according to an embodiment of the present disclosure, shown in front, top, and right-side views.

FIG. 19: An assembly according to another embodiment of the present disclosure, shown in front, top, and right-side views.

FIG. 20: An assembly according to another embodiment of the present disclosure, shown in front, top, and right-side views.

FIG. 21: An assembly according to another embodiment of the present disclosure, shown in front, top, and right-side views.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +/−5% or less, +/−1% or less, and +/−0.1% or less of and from the specified value), insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, the terms “including,” “containing,” and “comprising” are used in their open, non-limiting sense.

As used in this disclosure, the singular forms include the plural forms and vice versa unless the context clearly indicates otherwise.

The articles “a” and “an” are used in this disclosure to refer to one or more than one (i.e., to at least one) of the grammatical object of the article. By way of example, “an element” means one element or more than one element.

As used herein, unless otherwise stated or indicated, “s” refers to second(s), “min” refers to minute(s), and “h” refers to hour(s).

The present disclosure provides a method for aligning fibrils (e.g., protein fibrils, such as, for example, collagen fibrils) in a three-dimensional matrix. The resulting matrix may have a variety of uses, including, but not limited to, cell culture, tissue mimetics, cell differentiation, or as a drug screening substrate.

In an aspect, the present disclosure provides a method for aligning fibrils (e.g., protein fibrils, such as, for example, collagen fibrils). The fibrils are at least partially aligned. The alignment may be described via coherence index (CI). CI is a range of 0 to 1, where 0 corresponds to randomly oriented fibrils, and 1 corresponds to perfect alignment. The fibrils aligned via a method of the present disclosure may have a CI greater than or equal to 0.1 but less than 1, greater than or equal to 0.11 but less than 1, greater than or equal to 0.12 but less than 1, greater than or equal to 0.13 but less than 1, greater than or equal to 0.14 but less than 1, greater than or equal to 0.15 but less than 1, greater than or equal to 0.16 but less than 1, greater than or equal to 0.17 but less than 1, greater than or equal to 0.18 but less than 1, greater than or equal to 0.19, or greater than or equal to 0.2 but less than 1 but less than 1.

A method of the present disclosure comprises disposing a reconstituted protein (e.g., a disaggregated protein) on a substrate. The substrate may be arranged on surface that is oblique to a flat and/or level surface. The substrate may be arranged prior to or after the reconstituted protein is disposed on the substrate. While the substrate is arranged on an oblique surface, fibrillization of the reconstituted protein is initiated. Following fibrillization, the fibrils have a coherence index (CI) of greater than or equal to 0.1 but less than 1, greater than or equal to 0.11 but less than 1, greater than or equal to 0.12 but less than 1, greater than or equal to 0.13 but less than 1, greater than or equal to 0.14 but less than 1, greater than or equal to 0.15 but less than 1, greater than or equal to 0.16 but less than 1, greater than or equal to 0.17 but less than 1, greater than or equal to 0.18 but less than 1, greater than or equal to 0.19, or greater than or equal to 0.2 but less than 1 but less than 1.

A method of the present disclosure comprises disposing the disaggregated protein (e.g., reconstituted collagen) on a substrate. The substrate may be arranged on surface that is oblique to a flat and/or level surface. The substrate may be arranged prior to or after the reconstituted collagen is disposed on the substrate. CI may be used as an objective measure to quantify fibril alignment. While the substrate is arranged on an oblique surface, fibrillization of the disaggregated protein (e.g., reconstituted collagen) is initiated. Following fibrillization, the collagen fibrils have a coherence index (CI) of greater than or equal to 0.1 but less than 1, greater than or equal to 0.11 but less than 1, greater than or equal to 0.12 but less than 1, greater than or equal to 0.13 but less than 1, greater than or equal to 0.14 but less than 1, greater than or equal to 0.15 but less than 1, greater than or equal to 0.16 but less than 1, greater than or equal to 0.17 but less than 1, greater than or equal to 0.18 but less than 1, greater than or equal to 0.19, or greater than or equal to 0.2 but less than 1 but less than 1.

Various substrates may be used. The substrate may have a flat, solid, and/or non-porous surface upon which the disaggregated protein is disposed. For example, the substrate may be metal, glass, plastic, or any combination of these or other materials. In various examples, the substrate may have one or more flat surfaces. In various examples, the substrate is a glass slide or glass coverslip.

The substrate optionally has one or more coatings disposed thereon. The coating may be glutaraldehyde, a glutaraldehyde-based coating, a co-polymer, such as, for example poly(styrene-alt-maleic anhydride), or any combination thereof. The coating may have a thickness of 3 to 20 nm, including all 0.1 nm values and ranges therebetween (e.g., 3, 4, 5, 6, 7, 8, 9, 10, 11, 12, 13, 14, 15, 16, 17, 18, 19, or 20).

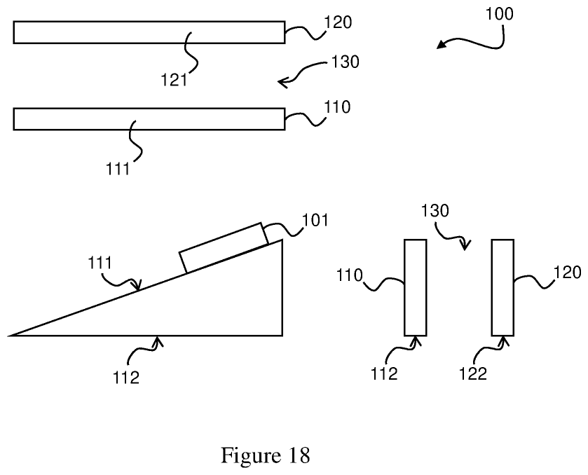

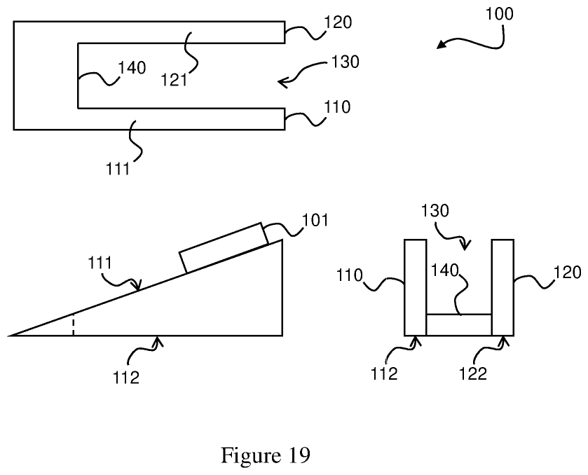

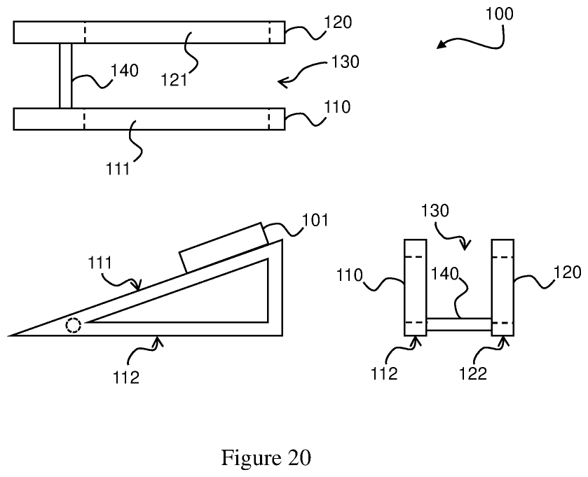

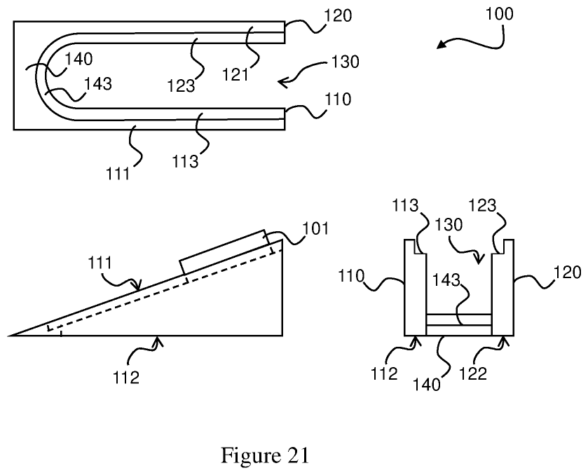

The disaggregated proteins and/or substrate may be disposed on a surface that is oblique relative to the plane of the horizon (i.e., oblique to a level surface). The surface may be on an assembly (e.g., an assembly 100 shown in FIGS. 18 to 21), such that the substrate is disposed on a flat surface of the assembly. In various examples, the assembly 100 comprises a first structure 110 and a second structure 120. For example, the first structure 110 may be a polygonal structure and/or the second structure 120 may be a polygonal structure. Each of the first structure 110 and the second structure 120 may have a substrate surface 111, 121—a surface upon which the substrate or workpiece 101 may be disposed. The substrate surfaces 111, 121 of the first structure 110 and the second structure 120 may be coplanar. The substrate surfaces 111, 121 may be smooth or rough. For example, a rough surface may have an increased coefficient of static friction compared to a smooth surface, which can impede the substrate or workpiece 101 from sliding down the substrate surfaces 111, 121. The substrate surfaces 111, 121 of the first structure 110 and the second structure 120 may be at least partially separated from each other by a gap 130 (i.e., spaced apart from each other). In other words, the assembly 100 may have a gap 130 at least partially dividing the substrate surfaces 111, 121. The first structure 110 and the second structure 120 may have a base surface 112, 122, in which the substrate surfaces 111, 121, are oblique relative to the base surfaces 112, 122. The base surface 112, 122 may be configured to support the assembly 100 on a tabletop or other surface, and the assembly 100 may be configured to hold a work piece 101. In the example shown in FIG. 18, the first structure 110 and the second structure 120 are separate elements of the assembly 100 separately arranged together to support the work piece 101. In another example shown in FIG. 19, the assembly 100 is a monolithic structure, wherein the first structure 110 and the second structure 120 are adjoined by a connecting structure 140 as a unitary element that is configured to hold a work piece 101. In another example shown in FIG. 20, the connecting structure 140 may be a separate element from the first structure 110 and the second structure 140, which are arranged to hold the work piece 101. The first structure 110 and the second structure 120 may be solid elements (as shown in FIG. 18) or hollow frame elements (as shown in FIG. 20). In the example shown in FIG. 21, the first structure 110 and the second structure 120 may comprise grooves 113, 123 at the substrate surfaces 111, 121. The grooves 113, 123 may be open to the gap 130. The grooves 113, 123 may be configured to receive the work piece 101, so as to retain the substrate on the substrate surfaces 111, 121. In some embodiments, the grooves 113, 123 may connect via a connecting groove 143 of the connecting structure 140, thereby forming a continuous groove that may retain the work piece 101 on the assembly. In various examples, the assembly 100 comprises the substrate. For example, the assembly 100 may be the substrate and the substrate surfaces 111, 121 of the assembly 100 may be the surfaces on which the disaggregated proteins are disposed. The work piece 101 may be the substrate.

The oblique surface is oblique relative to a flat and level surface (e.g., a level surface). The angle may be greater than 0° but less than 90°. For example, the angle may be 0.1° to 45°, including every 0.1° value and range therebetween, relative to a flat and level surface. In various embodiments, the angle is 7.5° to 30°, including every 0.1° value and range therebetween (e.g., 7.5°, 15°, 22.5°, or 30°).

Various fibrils may be aligned by a method of the present disclosure. For example, fibrils that form hydrogels with porous structures may be aligned. For example, the fibrils may be protein fibrils. The proteins self-assemble/self-associate/aggregate to form fibrils. The assembled proteins are bound together via non-covalent interactions (e.g., Coulombic interactions, hydrophobic interactions, π-π interactions, and the like, and combinations thereof) and van der Waals interactions. These protein fibrils may further form an entangled network of fibers that restrict the flow of water to form a hydrogel. That is, a hydrogel of the present disclosure may comprise water and fibril of the present disclosure, where the fibrils are aligned as described herein. In an embodiment, the hydrogel comprises crosslinked fibrils. In an embodiment, the hydrogel comprises only non-covalently crosslinked fibers and no chemically crosslinked fibers. In an embodiment, the fibers non-covalently associate via one or more non-covalent interactions (e.g., hydrophobic interactions, π-π interactions, hydrogen bonds, and the like, and combinations thereof). The protein may be collagen, fibrinogen, silk fibroin, or any combination thereof. Various types of collagen may be used (e.g., types I, II, III, IV, or any combination thereof). In various examples, the collagen is collagen type I. The reconstituted collage may be disposed at a concentration of 1 to 10 mg/mL, including all 0.1 mg/mL values and ranges therebetween (e.g., 1, 2, 3, 4, 5, 6, 7, 8, 9, or 10 mg/mL) (e.g., 2 mg/mL or about 2 mg/mL).

The proteins and/or protein fibrils may be crosslinked. Various crosslinking methods are known in the art. For example, the crosslinking may be chemical crosslinking via the use of a crosslinking agents. The crosslinking may result in intermolecular covalent bonds between one or more proteins. Examples of crosslinking agents include, but are not limited to, carbodiimide crosslinking agents or other agents that create an activated ester that is susceptible to reaction with a suitable nucleophile (e.g., an amine, a thiol) or other reagents used to form an amide bond (e.g., reagents typically used in solid-phase peptide synthesis to link amino acid residues). The suitable nucleophile may be a nucleophilic sidechain of an amino acid residue or the N-terminus of a protein. The activated ester may be formed from a carboxylic acid of the protein, such as, for example, the carboxylic acid of a glutamic acid side chain, an aspartic acid side chain, or C-terminus of the protein. For example, the crosslink may be an amide bond or thioester bond. Suitable crosslinking agents include, but are N,N′-dicyclohexylcarbodiimide (DCC), N,N′-diisopropylcarbodiimide (DIC), 1-ethyl-3-(3-dimethylaminopropyl)carbodiimide (EDC), and the like. Other crosslinking reagents include, but are not limited to, hydroxybenzotriazole (HOBt), hexafluorophosphate benzotriazole tetramethyl uronium (HBTU), hexafluorophosphate azabenzotriazole tetramethyl uronium (HATU), benzotriazol-1-yloxytris(dimethylamino)phosphonium hexafluorophosphate (BOP), and benzotriazol-1-yloxytripyrrolidinophosphonium hexafluorophosphate (PyBOP), and the like. Additional examples of crosslinking agents include, but are not limited to, genipin, glutaraldehyde, or a one or more carbohydrates (e.g., the crosslinking may be done via glycosylation). Crosslinking may occur prior to fibrillization, during fibrillization, after fibrillization, before gelation, and/or after gelation.

The fibrils aligned by a method of the present disclosure may have a desirable CI. For example, the CI may be 0.1 to 0.5, including all 0.01 values and ranges therebetween (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50) (e.g., 0.15 to 0.5 or 0.2 to 0.5). Without intending to be bound by any particular theory, it is considered as the angle of the oblique surface is increased, the CI also increases.

The protein may be fibrillized after the substrate upon which the protein is disposed is arranged onto an oblique surface. Fibrillization or initiation of fibrillization may be achieved by various methods known in the art. For example, when the protein is collagen, fibrillization may be initiated by heating the reconstituted collagen to about 37° C. or 37° C. at about 95% humidity or 95% humidity and in an atmosphere comprising about 5% CO₂ or 5% CO₂. In various other examples, fibrillization may be initiated by seeding (e.g., contacting the disaggregated protein with a “seed” protein fibril). The “seed” protein fibril may act as a template by which other disaggregated proteins self-assemble such that a protein fibril is formed.

In an aspect, the present disclosure provides a three-dimensional hydrogel matrix. For example, the hydrogel matrix comprises one or more proteins that self-assemble to form protein fibrils. For example, the protein may be collagen (e.g., type I collagen, type II collagen, type III collagen, type IV collagen, or any combination thereof). The hydrogen matrix may be made by a method of the present disclosure.

The hydrogel matrix comprises water and a plurality of protein fibrils. The hydrogel matrix can comprise various amounts of water. In various examples, a hydrogel comprises 80 to 99%, such as 85 to 99.9% by weight (based on the total weight of the composition) water. In an embodiment, the hydrogel matrix comprises about 91 to 99.9% by weight water. In various embodiments, the hydrogel matrix comprises 99 to 99.5% weight water.

The three-dimensional matrix (e.g., collagen matrix) may have various desirable properties. The protein fibrils (e.g., collagen fibrils) are at least partially aligned and have a coherence index (CI) of greater than or equal to 0.1 but less than 1, greater than or equal to 0.11 but less than 1, greater than or equal to 0.12 but less than 1, greater than or equal to 0.13 but less than 1, greater than or equal to 0.14 but less than 1, greater than or equal to 0.15 but less than 1, greater than or equal to 0.16 but less than 1, greater than or equal to 0.17 but less than 1, greater than or equal to 0.18 but less than 1, greater than or equal to 0.19, or greater than or equal to 0.2 but less than 1 but less than 1. For example, the CI of the aligned protein fibrils (e.g., collagen) may be 0.1 to 0.5, including all 0.01 values and ranges therebetween (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50) (e.g., 0.15 to 0.5 or 0.2 to 0.5). The matrix may be porous. The matrix may have an average pore diameter of ~6 to 10 μm, including all 0.1 μm values and ranges therebetween. Pore size as referred to herein refers to 50% of total pore area as the mean pore diameter. The fibrils may have an average fibril diameter of 0.4 to 1.5 μm, including all nm values and ranges therebetween (e.g., 0.65 to 0.725 μm). The hydrogel matrix may have an elastic modulus greater than that of collagen aligned on a 0° surface. For example, the elastic modulus may be greater than or equal to 105 Pa. Without intending to be bound by any particular theory, it is considered that the elastic modulus increases as the CI increases. Without intending to be bound by any particular theory, it is considered that fibril size can be controlled by modifying

The hydrogel matrix may comprise crosslinked protein fibrils and/or crosslinked proteins. For example, the crosslinking may be chemical crosslinking via the use of a crosslinking agents. The crosslinking may result in intermolecular covalent bonds between one or more proteins. Examples of crosslinking agents include, but are not limited to, carbodiimide crosslinking agents or other agents that create an activated ester that is susceptible to reaction with a suitable nucleophile (e.g., an amine, a thiol) or other reagents used to form an amide bond (e.g., reagents typically used in solid-phase peptide synthesis to link amino acid residues). The suitable nucleophile may be a nucleophilic sidechain of an amino acid residue or the N-terminus of a protein. The activated ester may be formed from a carboxylic acid of the protein, such as, for example, the carboxylic acid of a glutamic acid side chain, an aspartic acid side chain, or C-terminus of the protein. For example, the crosslink may be an amide bond or thioester bond. Suitable crosslinking agents include, but are N,N′-dicyclohexylcarbodiimide (DCC), N,N′-diisopropylcarbodiimide (DIC), 1-ethyl-3-(3-dimethylaminopropyl)carbodiimide (EDC), and the like. Other crosslinking reagents include, but are not limited to, hydroxybenzotriazole (HOBt), hexafluorophosphate benzotriazole tetramethyl uronium (HBTU), hexafluorophosphate azabenzotriazole tetramethyl uronium (HATU), benzotriazol-1-yloxytris(dimethylamino)phosphonium hexafluorophosphate (BOP), and benzotriazol-1-yloxytripyrrolidinophosphonium hexafluorophosphate (PyBOP), and the like. Additional examples of crosslinking agents include, but are not limited to, genipin, glutaraldehyde, or a one or more carbohydrates (e.g., the crosslinking may be done via glycosylation). Crosslinking may occur prior to fibrillization, during fibrillization, after fibrillization, before gelation, and/or after gelation.

In an aspect, the present disclosure provides uses for the hydrogel matrix (e.g., collagen matrix) of the present disclosure. For example, the hydrogel matrix (e.g., collagen matrix) may be used as a cell culture matrix, as a substrate for drug screening, or as a tissue mimetic.

In various examples, the hydrogel matrix can be used for drug screening. For example, a drug of interest may be contacted with a population of cells to determine the efficacy or any other potential effects on the population of cells encased in the matrix.

In various examples, the hydrogel matrix can be used as a tissue mimetic. For example, such a use includes uses for material design and disease modeling.

For example, when used as a cell culture matrix, the collagen matrix may allow and/or induce cellular differentiation. For example, an activator may be added in combination with a plurality of cells, which may be referred to as treating the cells with an activator. In various examples, cellular differentiation may occur in the absence of an activator via cell contractility. When the cells are treated with an activator, they may differentiate.

For example, the matrix may allow fibroblast differentiation when the fibroblasts are cultured in a matrix, where the matrix has a CI based on the alignment of fibrils at an angle of 15° to 35° (e.g., 15°, 16°, 17°, 18°, 19°, 20°, 21°, 22°, 23°, 24°, 25°, 26°, 27°, 28°, 29°, 30°, 31°, 32°, 33°, 34°, or 35°). Fibroblasts cultured from a medium having such a CI have similar RNA profiles to myofibroblasts or fibroblasts cultured in combination with TGF-β1.

For example, the matrix may allow macrophage differentiation. For example, the matrix may be used for the polarization of uncommitted macrophages (M0) into an anti-inflammatory phenotype (M2). This may occur after the addition of an activator, such as, for example, phorbol 12-myristate 13-acetate (PMA).

In various examples, the matrix may comprise crosslinked proteins and/or crosslinked protein fibrils.

In an aspect, the present disclosure provides an assembly 100 upon which substrates 101 may be arranged, as shown in FIGS. 18-21.

The assembly 100 includes a first structure 110 and a second structure 120—for example, a first polygonal structure and a second polygonal structure. The first structure 110 and the second structure 120 are at least partially separated (i.e., divided) by a gap 130, and wherein the first structure 110 and the second structure 120 have a base surface 112, 122 and an oblique surface 111, 121 relative to the base surface 112, 122. The assembly 100 is configured to hold a work piece 101 (e.g., a petri dish or other substrate capable of cell growth or protein self-assembly). The gap 130 allows for improved access to the substrate 101 for the application of heat to the substrate 101. The assembly 100 may comprise a first structure 110 and a second structure 120 being separate elements (as shown in FIG. 18) or a monolithic structure connected by a connecting structure 140 (as shown in FIG. 19). The first structure 110 and the second structure 120 may be solid elements or hollow frame elements (as shown in FIG. 20). The first structure 110 and the second structure 120 may include grooves 113, 123 that are configured to retain the work piece 101 on the substrate surfaces 111, 121.

The assembly may be made by a variety of manufacturing techniques. For example, the assembly may be additive manufacturing (e.g., 3D printing).

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various embodiments of the present disclosure, they are not intended to be limiting in any way.

Statement 1. A method for aligning protein fibrils comprising: disposing a disaggregated protein capable of fibrillization on a substrate; arranging the substrate onto a surface that is oblique to a flat and level plane; initiating fibrillization of the disaggregated protein; wherein following fibrillization the protein fibrils have a coherence index (CI) of greater than or equal to 0.1 but less than 1, greater than or equal to 0.11 but less than 1, greater than or equal to 0.12 but less than 1, greater than or equal to 0.13 but less than 1, greater than or equal to 0.14 but less than 1, greater than or equal to 0.15 but less than 1, greater than or equal to 0.16 but less than 1, greater than or equal to 0.17 but less than 1, greater than or equal to 0.18 but less than 1, greater than or equal to 0.19, or greater than or equal to 0.2 but less than 1 but less than 1 (e.g., greater than or equal to 0.15 but less than 1 or greater than or equal to 0.2 but less than 1).
Statement 2. A method according to Statement 1, wherein the disaggregated protein is collagen (e.g., type I collagen).
Statement 3. A method according to Statement 1 or Statement 2, wherein the oblique substrate is oblique at an angle of 0.1° to 45°, including every 0.1° value and range therebetween, relative to a flat and level surface.
Statement 4. A method according to Statement 3, wherein the angle is 7.5° to 30°, including every 0.1° value and range therebetween (e.g., 7.5°, 15°, 22.5°, or 30°).
Statement 5. A method according to any one of the preceding Statements, wherein the CI is 0.1 to 0.5, including all 0.01 values and ranges therebetween (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50) (e.g., 0.15 to 0.5 or 0.2 to 0.5).
Statement 6. A method according to any one of the preceding Statements, wherein the initiating fibrillization comprises heating the reconstituted protein (e.g., collagen) at about 37° C. at about 95% humidity and in an atmosphere comprising about 5% CO₂.
Statement 7. A hydrogel matrix (e.g., collagen matrix) comprising a plurality of protein fibrils, wherein the collagen fibrils are at least partially aligned and have a coherence index (CI) of greater than or equal to 0.15 but less than 1 (e.g., greater than or equal to 0.2 but less than 1).
Statement 8. A hydrogel matrix (e.g., collagen matrix) according to Statement 7, wherein the collagen matrix has an average pore diameter of ~6 to 10 μm, including all 0.1 μm values and ranges therebetween.
Statement 9. A hydrogel matrix (e.g., collagen matrix) according to Statement 7 or Statement 8, wherein the collagen fibrils have an average fibril diameter of 0.65 to 0.725 μm, including all 0.001 μm values and ranges therebetween.
Statement 10. A hydrogel matrix (e.g., collagen matrix) according to any one of Statements 7 to 9, wherein the CI is 0.1 to 0.5, including all 0.01 values and ranges therebetween (e.g., 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, or 0.50) (e.g., 0.15 to 0.5 or 0.2 to 0.5).
Statement 11. A hydrogel matrix (e.g., collagen matrix) according to any one of Statements 7 to 10, wherein the collagen matrix has an elastic modulus of greater than 105 Pa.
Statement 12. A cell culture matrix comprising the collagen matrix according to any one of Statements 7 to 11.
Statement 13. A method for differentiating fibroblasts, comprising: disposing fibroblasts into the cell culture matrix according to Statement 12; optionally disposing TGF-β1 into the cell culture matrix; culturing the fibroblasts in the cell culture matrix; and optionally, isolating the differentiated fibroblasts. Without intending to be bound by any particular theory, it is considered that the cell contractility triggers the differentiation caused by the aligned matrix of protein fibrils.
Statement 14. An assembly including a first polygonal structure and a second polygonal structure, wherein the first polygonal structure and the second polygonal structure are divided or at least partially separated by a gap, and wherein the first polygonal structure and the second polygonal structure have a base and an oblique surface relative to the base, wherein the assembly is configured to hold a work piece (e.g., a petri dish or other substrate capable of cell growth or protein self-assembly).
Statement 15. A substrate for drug screening comprising the hydrogel matrix (e.g., collagen matrix) according to any one of Statements 7 to 11.
Statement 16. A tissue mimetic comprising the hydrogel matrix (e.g., collagen matrix) according to any one of Statements 7 to 11.
Statement 17. A method for differentiating cells comprising disposing cells into the cell culture matrix according to Statement 12; optionally disposing an activator into the cell culture matrix (e.g., phorbol 12-myristate 13-acetate (PMA), TGF-β1, and the like), culturing the cells in the cell culture matrix, and optionally isolating the differentiated cells.

The following examples are presented to illustrate the present disclosure. They are not intended to be limiting in any matter.

### Example 1

This example provides a description the methods of the present disclosure.

Collagen alignment is one of the key microarchitectural signatures of many pathological conditions, including scarring and fibrosis. Investigating how collagen alignment modulates cellular functions will pave the way for understanding tissue scarring and regeneration, as well as new therapeutic strategies. However, current approaches for the fabrication of three-dimensional (3D) aligned collagen matrices are low-throughput and require special devices. To overcome these limitations, an approach to reconstitute homogenous 3D collagen matrices with adjustable degree of fibril alignment using 3D printed inclined surfaces was developed and is described herein. By characterizing the mechanical properties of reconstituted matrices, it was found that the elastic modulus of collagen matrices is enhanced with an increase in the alignment degree. The reconstituted matrices were used to study fibroblast behavior to reveal the progression of scar formation where a gradual enhancement of collagen alignment can be observed. It was found that matrices with aligned fibrils trigger fibroblast differentiation into myofibroblasts via cell contractility, while collagen stiffening through a crosslinker did not. These results suggest the impact of collagen fibril organization on the regulation of fibroblast differentiation. Overall, this approach to reconstitute 3D collagen matrices with fibril alignment opens opportunities for biomimetic pathological-relevant tissue in vitro, which can be applied for other biomedical research.

In this study, a reproducible and well-defined 3D collagen matrix was established that allows the mimicry of progression of tissue fibrosis where a gradual enhancement of collagen fibril alignment can be observed. The effects of collagen alignment of various degrees on human dermal fibroblasts were subsequently investigated. Cells were assessed at various resolutions using RNA sequencing, protein expression and functional analysis. The described biomimetic model is robust and simple, high-throughput, and most importantly noninvasive, thus leaving no residual component within the matrices, which can be applied to other biomedical applications.

Results and discussion. Aligned collagen microarchitecture is known as a pathologically relevant extracellular matrix characteristic. Various methods have been devised to fabricate matrices with aligned collagen fibrils, namely, using microfluidic devices, paramagnetic microbeads, cyclic mechanical stretch devices, constrained cellular compaction, and microextrusion 3D printing techniques. However, reported techniques are either inherently invasive and/or require special devices that essentially reduce reproducibility across research groups. In addition, they generally do not allow for high-throughput reconstitution of homogenous matrices, which allows for the study of downstream cell signaling. The comparison of various techniques for reconstituting 3D collagen matrices with fibril alignment is provided in Table 2. The present disclosure overcomes all these current limitations and further used the reconstituted matrices to reveal the progression of tissue fibrosis, where a gradual enhancement of collagen alignment can be observed, and its effects on fibroblast behavior as a result of these microarchitectural changes.

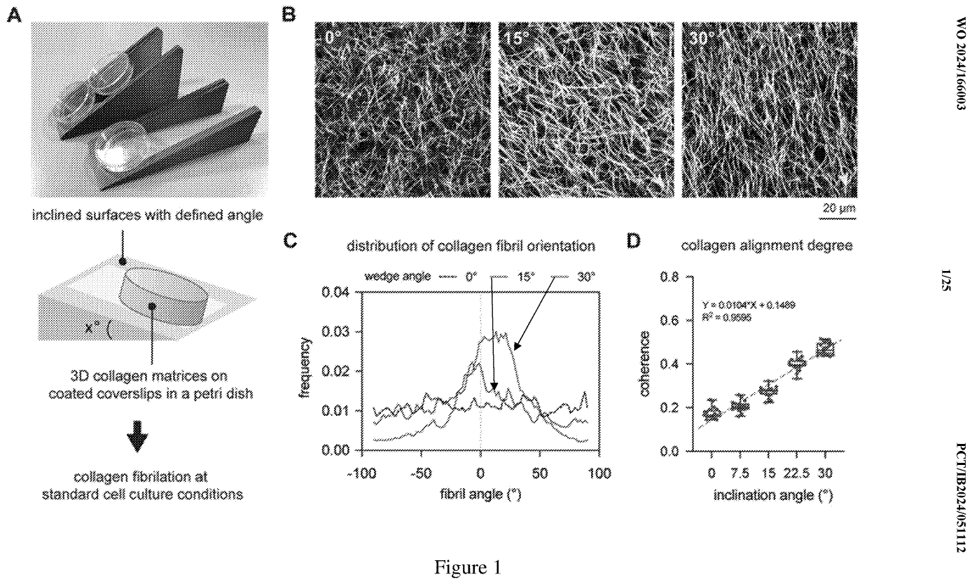

Reconstitution and characterization of 3D collagen matrices with adjustable fibril alignment degree. To reconstitute 3D collagen matrices with aligned collagen fibrils, collagen solutions were prepared and transferred onto coated coverslips. During collagen fibrillation, coverslips were placed onto a planar surface (0°) or onto printed inclined surfaces with angles of 7.5°, 15°, 22.5° and 30°, as depicted in FIG. 1A. The inclined surfaces have been designed to have a gap in the middle, allowing ample heat transfer for collagen fibrillation, as the fibrillation temperature is known to affect collagen microstructure. It is important to note that there is non-uniformity in the thickness of collagen matrices that are reconstituted on inclined surfaces with angles exceeding 40°. The mechanism of reconstitution of collagen alignment using our techniques relies on the sedimentation effects caused by gravitational forces acting on growing fibrils. As these fibrils continue to grow, they eventually become relatively large micron-sized objects with sedimenting properties, which ultimately results in their movement in the direction of gravitation and subsequent alignment.

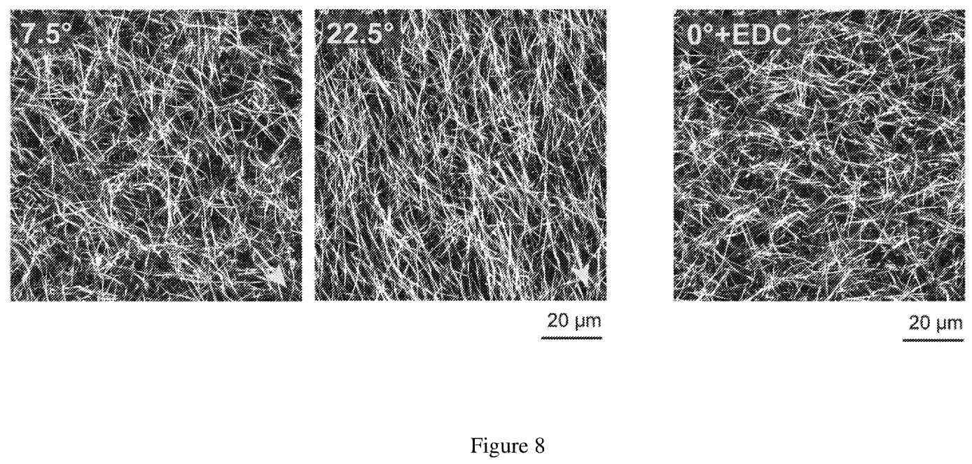

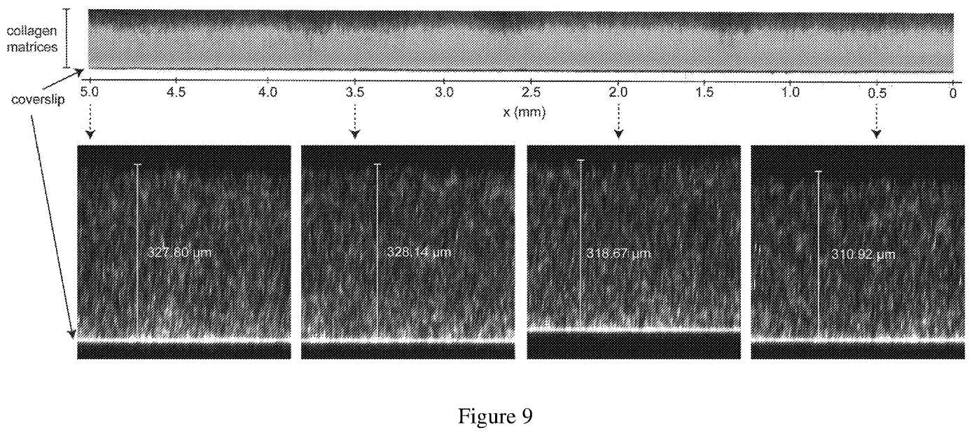

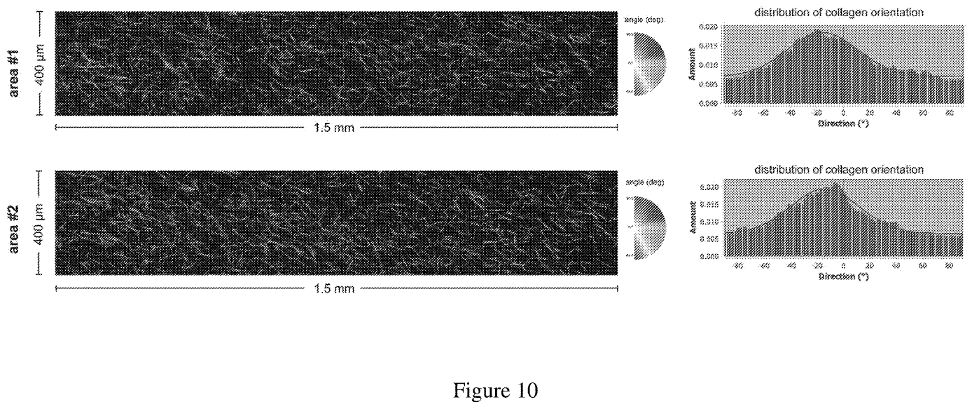

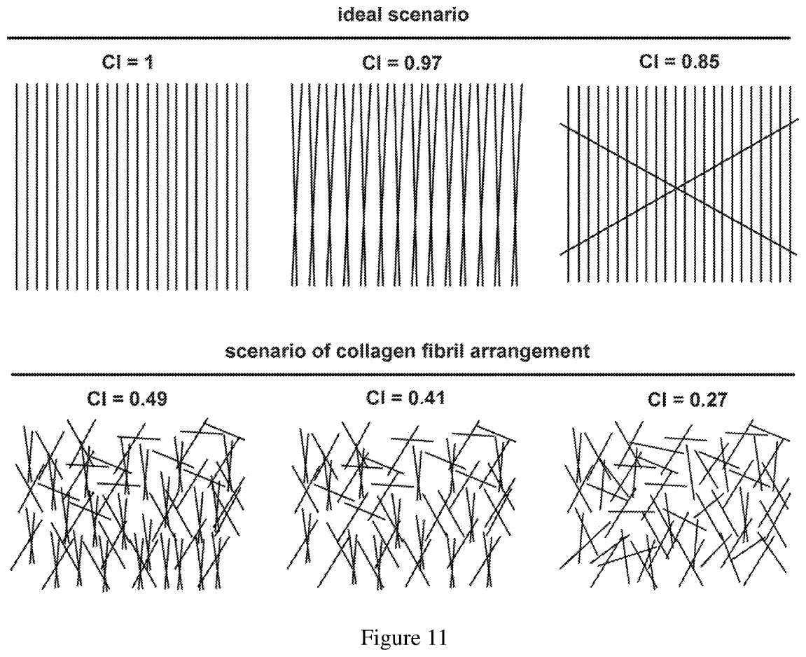

Upon collagen fibrillation, collagen matrices were fluorescently labeled by TAMRA-SE and visualized using a confocal laser scanning microscope. As shown in FIG. 1B, an increase in collagen alignment degree could be visually observed with an increase in the inclination angle. Representative images of collagen reconstituted on inclined surfaces with angles of 7° and 22.5° are depicted in FIG. 8. To show that the reconstituted collagen matrices from inclined surface with a 300 angle had uniform thickness (FIG. 9) and long-range fibril alignment (FIG. 10), the collagen network was imaged and quantified using a low-magnification objective. To further confirm the visual observation, the obtained images were quantitatively analyzed to gain insights into the distribution of collagen fibril orientation and their alignment degree. Analyzing collagen fibril orientation, collagen matrices fibrillated on inclined surfaces showed a Gaussian distribution, and the distribution peak was enhanced with increased inclination angle, while collagen matrices fibrillated on planar surfaces had uniformly distributed fibril orientation as fibrils were randomly oriented (FIG. 1C). To further quantified the fiber alignment index by calculating the coherence index (CI), as it has been demonstrated to be an objective approach to quantify collagen alignment CI ranges between 0 and 1, where 0 represents a perfectly random distribution of fibrils, while an index of 1 represents perfect fibril alignment. As shown in FIG. 1D, the CI of collagen matrices fibrillated on planar surfaces was 0.18±0.03, while collagen matrices fibrillated on inclined surfaces showed an increase in CI from 0.21±0.03 and 0.47±0.03 for inclination angles of 7.5° and 30°, respectively. The CI is proportional to the inclination angle, as shown by linear fitting (FIG. 1D, gray dashed line, R²=0.9595). It is important to note that the process of collagen fibrillation involves the self-assembly of collagen monomers and the entanglement of fibrils to form a stable 3D network and cannot achieve the level of alignment degree that can be produced using bioprinting techniques. To ensure that the lower CI values were not biased by the analysis methods, phantom images of ideal and collagen fibril arrangements were created and analyzed regarding their CI (FIG. 11). Analysis of their CI revealed that increasing the number of aligned fibrils while keeping the number of random collagen fibrils constant resulted in a slight change in the CI value from 0.41 to 0.49 (FIG. 11), highlighting the low CI in the aligned collagen matrices of the present disclosure.

Overall, using inclined surfaces with different angles allowed for the mimic of the gradual change in randomly organized collagen fibrils towards aligned collagen fibrils, mimicking the progression of tissue fibrosis. Additionally, the CI analysis suggests that using the approach described herein, the collagen alignment degree can be fine-tuned to recapitulate human normal skin (CI: 0.21±0.10), normotrophic skin (CI: 0.41±0.13), hypertrophic skin (CI: 0.46±0.16), and keloid scar skin (CI: 0.44±0.15). Overall, approach using 3D printed inclined surfaces with different angles is able to reconstitute well-defined and homogenous collagen matrices with an adjustable fibril alignment degree, which is in the range of human normal skin tissue and scars.

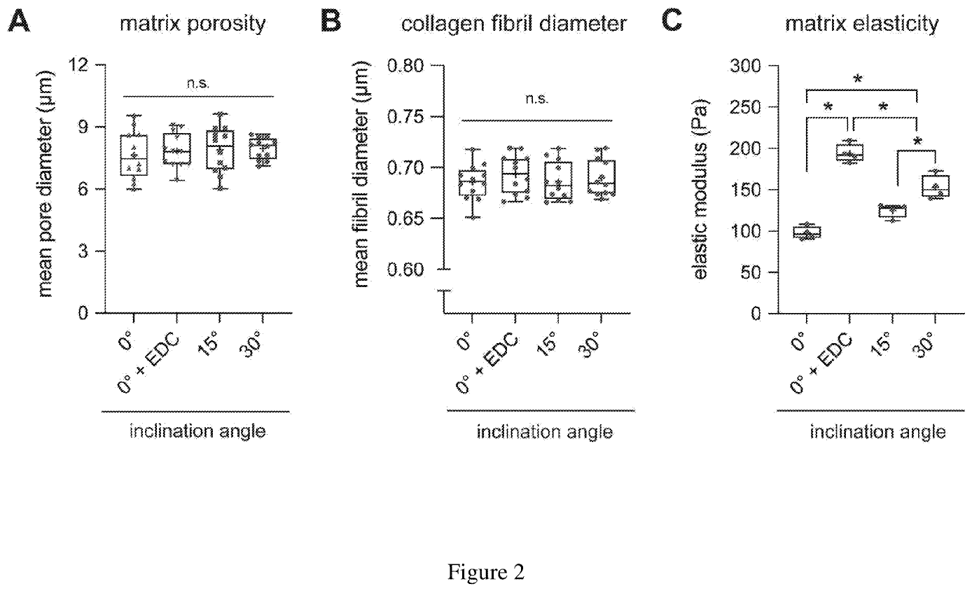

To determine whether the collagen alignment degree impacts fibroblast behavior, matrices reconstituted on a planar surface (0°) and inclined surfaces with 15° and 30° angles were chosen. Prior to doing that, the topological and mechanical properties of these reconstituted matrices were quantitatively characterized. Mean pore diameter and fibril diameter were analyzed using a custom-made image analysis toolbox, and both parameters were found to be consistent for all matrices and reconstitution conditions (FIGS. 2A and 2B). The bulk matrix elastic modulus was characterized using a non-destructive contactless rheometer. An increase in the elastic modulus was found with increasing alignment degree from 97.80±7.8 Pa to 124.7±8.4 Pa and 153±14.3 Pa for 0°, 15° and 30° inclination angles, respectively (FIG. 2C). These data are in line with other studies demonstrating that aligned collagen matrices exhibited a higher elastic modulus than collagen matrices with a random fibril distribution. It is known that matrix stiffness can modulate fibroblast functions. To decipher the impact of collagen alignment and matrix stiffness on fibroblast behavior, the stiffness of collagen matrices reconstituted on a planar surface was enhanced using a carbodiimide crosslinker (0°+EDC; E=193.8±11.3 Pa) while keeping the mean pore diameter (FIG. 2A) and fibril diameter (FIG. 2B) at levels similar to those of uncrosslinked and aligned matrices, as previously demonstrated. A representative image of EDC-crosslinked matrices is shown in FIG. 8. Interestingly, that although collagen matrices are crosslinked with EDC, their elastic modulus is relatively low and falls within the range of hundreds of Pascals. This is lower than the elastic modulus of native tissues, which contain additional ECM components. Nevertheless, even a small increase in collagen matrix elasticity has been demonstrated to have an impact on cell behavior in various cell types. In addition, anisotropy in microarchitecture of the collagen matrices will result in significantly differing bulk elastic properties when samples are measured longitudinal and perpendicular to collagen fibril alignment. It is expected that the longitudinal stiffness, parallel to the direction of alignment, will be considerably greater than perpendicular stiffness. Unfortunately, the current method used to measure the elastic modulus is unable to capture these differences. In our case, the measured modulus is to provide a comparison of elastic modulus between matrices.

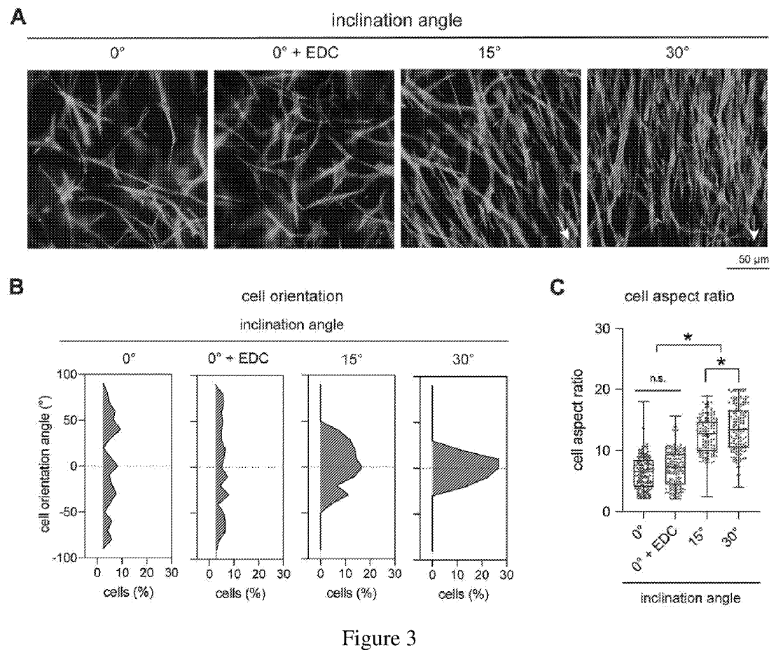

Collagen fibril alignment modulates the orientation and elongation of fibroblasts. It has been reported that cell orientation and elongation are altered by the organization of collagen fibrils. To demonstrate this aspect, primary human dermal fibroblasts were cultured onto the well-characterized 3D collagen matrices for 4 days. Afterwards, the cell cytoskeleton and nucleus were stained and visualized using an epifluorescence microscope. As shown in FIG. 3A, fibroblasts cultured on collagen matrices reconstituted on a planar surface (0°) were randomly oriented. An increase in the matrix stiffness using an EDC crosslinker did not affect cell orientation. However, fibroblasts cultured onto collagen matrices reconstituted at 15° and 30° inclination angles were collectively aligned. In addition, an enhancement was found in stress fibers in fibroblasts cultured onto matrices with aligned collagen fibrils when compared to cells cultured on randomly organized fibrils of both uncrosslinked and crosslinked conditions. Quantitative analysis of the cell orientation confirmed our visual observation (FIG. 3B). Interestingly, the orientation of cells cultured onto collagen matrices reconstituted on 15° collagen matrices appeared to be more dispersed when compared to the cells cultured onto the 30° counterpart. These data suggest that the orientation of the cell body is correlated with the collagen alignment degree. In addition to the analysis of cell orientation, the cell aspect ratio was quantified as an indication of cell elongation. As shown in FIG. 3C, it was found that cell elongation is modulated by the alignment degree of collagen fibrils. However, an increase in the bulk elastic modulus of the randomly distributed collagen fibrils (0°+EDC) does not affect cell elongation. These data suggest that the organization of collagen fibrils, but not matrix stiffness, modulates cell elongation in fibroblasts.

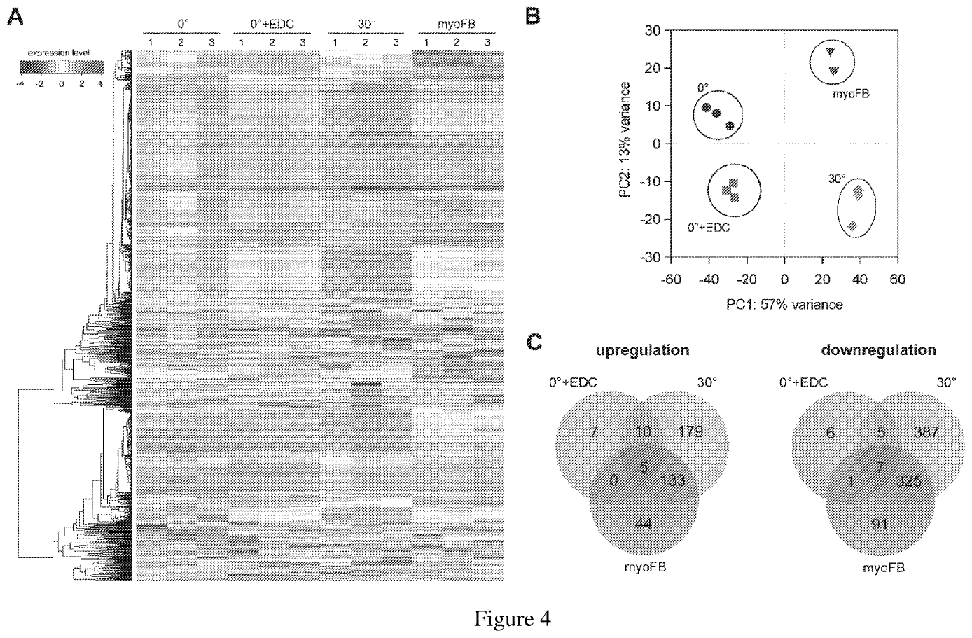

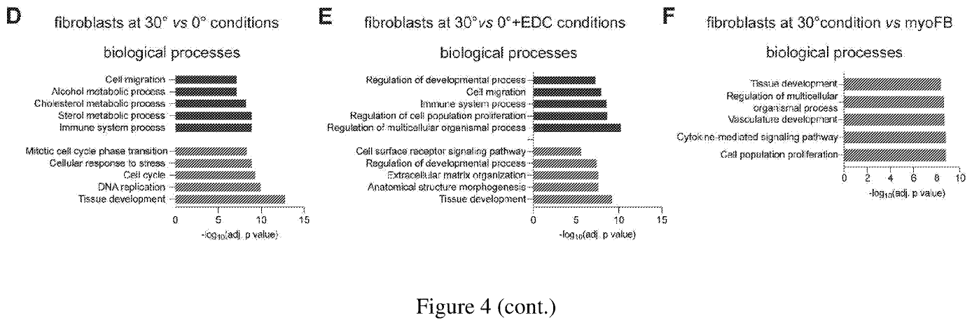

RNA sequencing reveals collagen alignment-mediated changes in the transcriptome of fibroblasts towards myofibroblasts. As demonstrated above, fibroblasts adapted their cell orientation and spreading behavior in response to collagen fibril alignment (FIG. 3). To reveal to what extent collagen fibril alignment affects fibroblast differentiation and functions, RNA sequencing of fibroblasts cultured on 0°, 0°+EDC, 30° and myofibroblasts (MyoFBs) was performed. In this case, fibroblasts were cultured onto a random matrix (0°) in the presence of TGF-β1 to differentiate them into myofibroblasts and were used as a control. As shown in FIG. 4A, it was found that the gene expression patterns of fibroblasts cultured on randomly distributed collagen fibrils (both 0° and 0°+EDC) were similar, while fibroblasts cultured onto an aligned fibril matrix (30°) demonstrated a transcriptome profile similar to that of myofibroblasts. Principal component analysis (PCA) confirmed that there is a clear difference between cells cultivated on random (00 and 0°+EDC) and aligned matrices (30°), as demonstrated by PC1 (principal component 1) shown in FIG. 4B. PCA also revealed differences in the transcriptomes of fibroblasts on an aligned matrix (30°) and myofibroblasts. From the RNASeq data, differentially expressed genes (DEGs) with a false discovery rate (FDR) cut-off of 0.05 and a minimal fold change of 2 for all conditions compared to cells cultured on a matrix with randomly distributed fibers without the EDC crosslinker (0°) were further analyzed. As shown in FIG. 4C, up- and downregulated DEGs were plotted as a Venn diagram. Fibroblasts cultivated on matrices with randomly distributed fibers (both 0° and 0°+EDC), irrespective of the bulk matrix stiffness/crosslinking, demonstrated less change in DEGs, confirming a similar transcriptome profile of both conditions. Fibroblasts cultivated on matrices with aligned fibrils (30°), however, showed an increase in both upregulated and downregulated DEGs when compared to cells cultivated on random matrices (both 0° and 0°+EDC), indicating that fibroblast function changes when cultivated on matrices with aligned fibrils. Interestingly, fibroblasts on an aligned matrix (30°) share common DEGs with myofibroblasts, but both cell types still exhibit distinct DEGs. To investigate enrichment in biological pathways, gene set analysis using the generally applicable gene-set enrichment (GAGE) method was performed. This analysis allows us to unbiasedly predict the biological pathway independent of the identified DEGs, as DEGs depend heavily on the criteria set for the cut-off, in our case an FDR cut-off of 0.05 and a minimal fold change of 2. The GAGE analysis allowed us to overcome these issues by using pathway knowledge; therefore, small but coordinated changes in the gene set can be used to predict the up- or downregulation of specific biological pathways. By doing this, the enriched biological pathways of cells in matrices with random fibrils (00 and 0°+EDC) were analyzed; no significant enrichment in specific pathways was found, as also suggested by minimal numbers of DEGs (FIG. 4C). By comparing fibroblasts cultured on matrices with aligned fibrils (30°) to (both 0° and 0°+EDC), a common downregulation in biological pathways associated with cell migration (FIGS. 4D and 4E) was found. In addition, gene sets in tissue development pathways have been shown to be upregulated in aligned matrices when compared to cells cultivated on matrices with random fibrils (00 and 0°+EDC). It also appears that fibroblasts cultured in matrix with aligned fibrils (30°) exhibited higher gene sets associated with cell proliferation when compared to matrices with random fibrils (0°) and enhanced expression of gene sets associated with matrix organization when compared to crosslinked matrices with random fibrils (0°+EDC). All these gene sets indicated the differentiation of fibroblasts into myofibroblasts, as they are nonmotile, contractile, and highly proliferative cells. In addition, fibroblasts cultured on aligned matrix possessed a transcriptome profile similar to that of myofibroblasts. By comparing their biological pathways using GAGE, an enrichment was found in gene sets associated with tissue development, regulation of multicellular organismal processes, cytokine-mediated signaling pathways and cell population proliferation (FIG. 4F), suggesting a higher cellular activity of fibroblasts in the aligned matrix.

In sum, the transcriptome analysis using RNA sequencing suggests that stiffening of the random matrix using a crosslinker minimally affected the transcriptome profile of fibroblasts, whereby matrix alignment appears to instruct fibroblast differentiation into myofibroblasts, as demonstrated by a similar transcriptome profile and enhanced expression of gene sets associated with myofibroblasts.

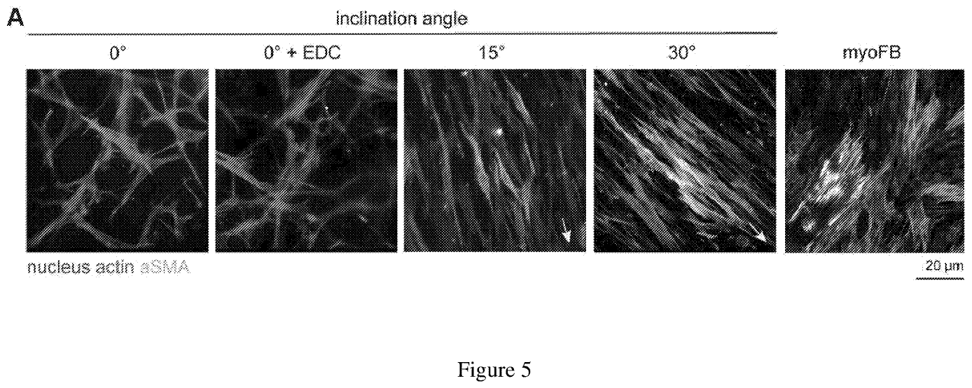

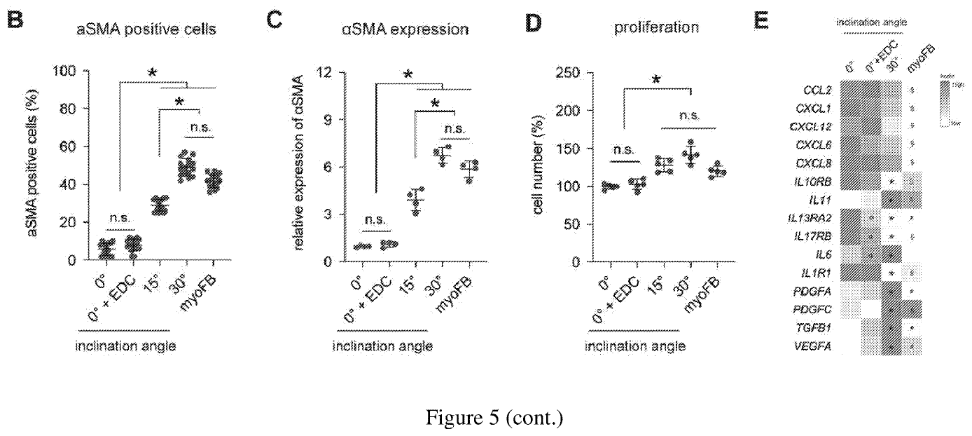

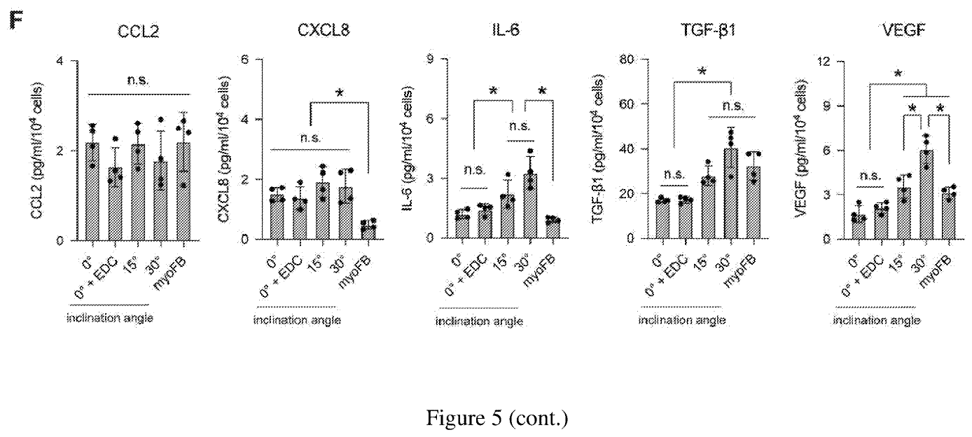

Aligned matrices instruct fibroblast differentiation but modulate distinct cytokine expression profiles. From transcriptome analysis, it was hypothesized that matrix alignment might trigger fibroblast differentiation, as clued by enhanced expression of gene sets in proliferation and matrix remodeling, which are incidentally reported characteristics and found in the myofibroblast transcriptome (FIG. 4). To address this, cells were stained with anti-αSMA antibody and manually quantified the obtained images regarding the number of αSMA-positive cells. αSMA expression is a well-known marker for myofibroblasts. FIG. 5A shows representative images of immunocytochemical staining of nuclei (blue), actin (red) and αSMA (gray). It could be visually observed that cells cultivated on aligned matrices, both 15° and 30°, presented αSMA expression similar to that of myofibroblasts, while it could not be observed in fibroblasts cultured on both random fibrils (0° and 0°+EDC). The number of αSMA-positive cells was quantified from the obtained images. As shown in FIG. 5B, an increase in αSMA cells was found in aligned matrices (both 15° and 30°) when compared to random matrices (0° and 0°+EDC) independent of matrix stiffening. Quantitative image analysis confirmed the visual observation. Additionally, the number of αSMA-positive cells increased with increasing alignment degree. Cells cultured on highly aligned collagen (30°) appear to show a slight increase, albeit without significant difference, in αSMA-positive cells when compared to myofibroblasts. Similarly, an increase in αSMA gene expression was observed as the inclination angle increased (FIG. 5C). These data indicate that the degree of matrix alignment can modulate the level of fibroblast differentiation.

In addition to αSMA expression, cellular proliferation is another important feature of myofibroblasts, as reported elsewhere and evident from the transcriptome analyses described here. The proliferation of cells was analyzed by counting cells using flow cytometry. As shown in FIG. 5D, enhanced cell proliferation was found in cells cultivated onto aligned matrices (both 150 and 30°) and myofibroblasts when compared to random matrices (both 0° and 0°+EDC). These data indicate that an increase in matrix stiffness in random matrices (0°+EDC vs 0°) does not affect cell proliferation in fibroblasts. In addition, it appears that fibroblasts cultivated onto an aligned matrix (30°) exhibited a slightly higher cell number than myofibroblasts, highlighting higher proliferative activity, as shown in the transcriptome analysis (FIG. 4F). A slight increase, but not significant, in cell number could be observed with increasing alignment degree. Overall, along with αSMA expression, enhanced cell proliferation also supports the notion hypothesized herein that matrix alignment instructs the differentiation of fibroblasts into myofibroblasts.

To further reveal functional differences of fibroblasts in random and aligned matrices to a greater extent, the gene expression of cytokines was analyzed based on our RNA sequencing data. As shown in FIG. 5E, a heat map of the gene expression of major cytokines involved in cell-cell interactions was plotted. Random matrices with enhanced stiffness (0°+EDC vs 0°) significantly enhanced the gene expression of IL13RA2, IL17RB and IL6. Aligned matrices significantly increased the expression of IL10RB, IL11, IL1R1, PDGFA, PDGFC, TGFB1, and VEGF in fibroblasts compared with random matrices (both 0° and 0°+EDC). Interestingly, myofibroblasts appear to express less CCL2, CXCL1, CXCL12, CXCL6, and CXCL8 than fibroblasts on both random (00 and 0°+EDC) and aligned matrices (30°).

Confirmed the expression of specific cytokines was confirmed, namely, CCL2, CXCL8, IL-6, TGF-β1 and VEGF, using multiplex bead-based ELISA, as these cytokines are important for tissue fibrosis (FIG. 5F). For all conditions, no change in CCL2 was found, which is involved in monocyte recruitment. Myofibroblasts appeared to secrete less CXCL8 but maintained IL-6 levels similar to those of fibroblasts in random matrices (both 0° and 0° EDC). CXCL8 regulates the directional migration of leukocytes and diminishes wound contraction, while IL-6 is known as a fibrotic mediator and triggers collagen production. Cells cultured on aligned matrices (both 15° and 30°) significantly increased their secretion of IL-6, TGF-β1 and VEGF. In addition, those cytokines increase with the degree of alignment when comparing cells on 15° with 30°, but the increment is significant only for VEGF. TGF-β1 is involved in the differentiation of fibroblasts and maintenance of the myofibroblast phenotype and can enhance collagen production and matrix remodeling in combination with IL-6. VEGF, on the other hand, is important for initiating and directing angiogenesis, which is involved in tissue fibrosis and has been proposed as a therapeutic target along with TGF-β1 inhibitors to resolve fibrosis. As shown in FIG. 5F, IL-6, TGF-β1 (not significant) and VEGF are secreted more by fibroblasts in aligned collagen (30°) when compared to myofibroblasts, which helps to explain the accumulation of myofibroblasts and excessive production of ECM components in fibrotic tissues.

Overall, the αSMA expression analyses, proliferation studies, and cytokine secretion studies data manifest and confirmed the results from RNA-Seq that fibril organization within collagen matrices can instruct myofibroblast differentiation in dependence of alignment degree, which we also demonstrated using αSMA staining and gene expression, as well as enhanced cell proliferation. In addition, these data reveal that fibroblasts cultured onto aligned matrices show differential expression patterns and levels of cytokines compared to myofibroblasts activated via TGF-β1. This result indicates that the organization of collagen fibrils can modulate distinct cellular functions and their cytokine secretion profiles that in turn mediate communication with other cells in the microenvironment.

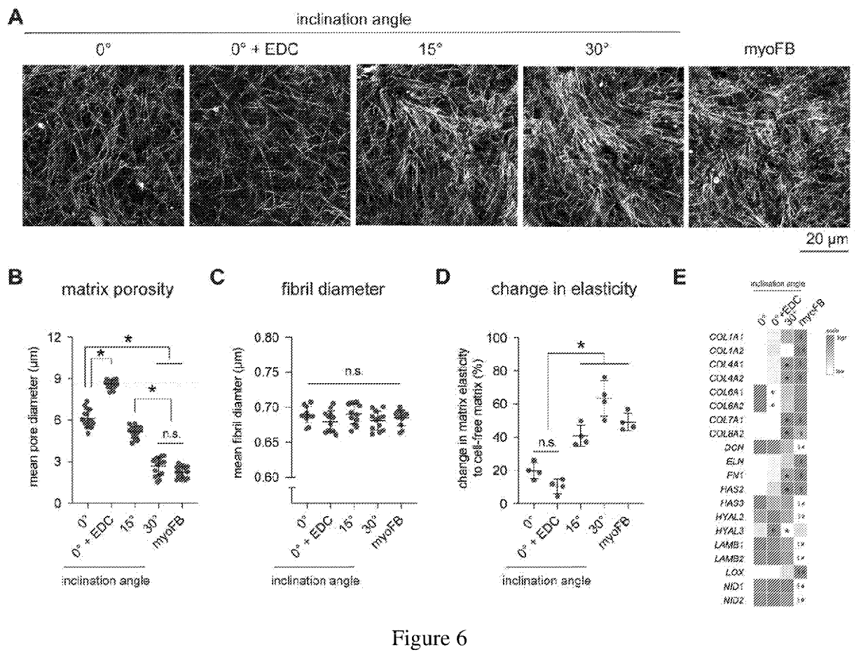

Matrix remodeling is enhanced in the aligned matrix. Another parameter that is an important measure of fibroblast function is the ability to perform matrix remodeling. To investigate this, first matrices were decellularized that housed fibroblasts for 3 days, visualized them using cLSM, and then quantified the mean pore and fibril diameters using the custom-built image analysis toolbox. Representative images of decellularized matrices under different conditions are shown in FIG. 6A. It could be visually observed that matrices of myofibroblasts and fibroblasts cultured on aligned matrices were strongly remodeled when compared to the collagen microarchitecture of random matrices. These data support that fibroblasts are triggered to differentiate in aligned collagen matrices, as myofibroblasts have been reported to exhibit a contractile phenotype that is capable of matrix remodeling. By analyzing the mean pore diameter, it was found that matrices remodeled by myofibroblasts showed a significant reduction in pore diameter compared to that of fibroblasts in random matrices (0° and 0° EDC), as shown in FIG. 6B. These data also demonstrated that the crosslinked matrices were minimally remodeled by fibroblasts. For aligned matrices, it was found that the reduction in pore diameter due to remodeling appeared to be dependent on the alignment degree and reached a pore diameter range similar to that of the matrix remodeled by myofibroblasts, correlating well with the number of αSMA-positive cells, as shown in FIG. 5B. As fibril diameter increases in the fibrotic tissues, the mean fibril diameter of the decellularized matrices was quantified. It was found that collagen fibril diameter remained similar in all matrix conditions, despite cell-mediated changes in mean pore diameter (FIG. 6C). It was previously shown that fibril diameter increases only in the presence of M2a macrophages in macrophage-fibroblast cocultures. In addition to the mean pore and fibril diameter, we analyzed the matrix elasticity using a contactless rheometer. To better compare the change in matrix elasticity, the data were normalized to the elasticity of cell-free matrices. As shown in FIG. 6D, matrices of myofibroblasts and fibroblasts cultured in aligned matrices showed significantly higher changes in matrix elastic modulus when compared to fibroblasts cultured in both random matrices (0° and 0°+EDC). It appears that the aligned matrices (30° only) showed slightly higher elastic modulus than matrices from myofibroblasts with similar topological properties (15°), however, with no significant differences. These data highlight the acceleration of ECM stiffening in fibrotic tissue.

As matrix stiffening could be modulated by physical changes in the ECM or excessive production of ECM components, we studied differences in the production of matrix components using data obtained from RNASeq. As shown in FIG. 6E, distinct expressions of matrix components in all matrix conditions are illustrated. It was shown that stiffening of the random matrix (0°+EDC) significantly reduced the expression of COL6A1 and COL6A2, while it enhanced the expression of HYAL3 when compared to the more compliant random matrices (0°). Collagen alignment triggered the expression of COL4A1, COL4A2, COL7A1, COL8A2, FN1, and HAS2 but a reduction in HYAL3 when compared to both random matrices (0° and 0° EDC). Interestingly, myofibroblasts expressed more COL1A1, COL1A2, ELN, and LOX but showed lower expression of DCN, HAS3, HYAL2, LAMB1, LAMB2, NID1, and NID2 than cells cultured on aligned matrices (30°). Although cells cultured on aligned matrices possessed characteristics of a myofibroblast phenotype, the expression of matrix components appears to be dependent on fibril orientation.

In sum, these data support that the fibroblasts differentiated through aligned matrices possess a similar degree of matrix remodeling capabilities as myofibroblasts activated through TGF-β1. Interestingly, matrix stiffness and orientation appear to regulate a distinct matrix component production regime by fibroblasts. These results suggest how specific microenvironments with specific ECM compositions and microarchitecture are made to assist the progression of disease, as well as maintain physiologically relevant conditions. For example, a gradual increase in collagen alignment might contribute to accelerating the tissue fibrotic condition by increasing matrix remodeling through cell-matrix interactions via enhanced expression of fibronectin, as previously reported.

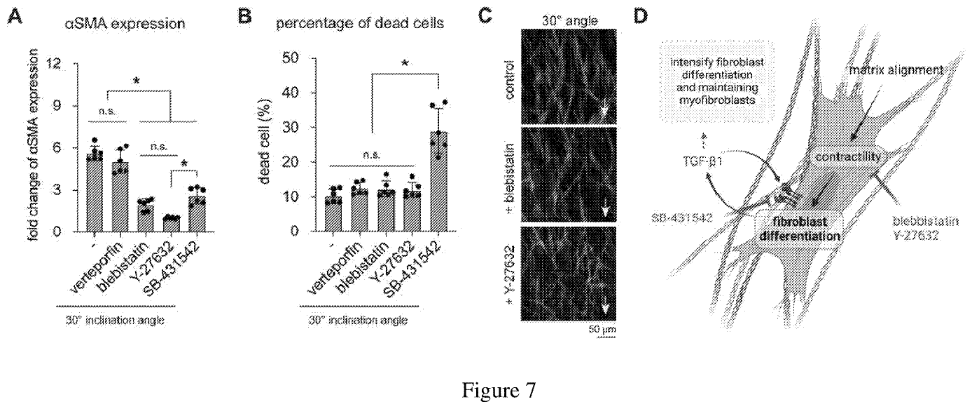

Cell contractility mediates fibroblast differentiation in aligned collagen matrices. Matrix alignment appears to affect fibroblast behavior in different aspects. In an attempt to understand the underlying signaling pathway related to fibroblast differentiation in aligned collagen matrices (30°), fibroblasts were treated with chemical inhibitors, namely, verteporfin (YAP inhibitor), blebbistatin (myosin inhibitor), Y-27632 (Rho kinase (ROCK) inhibitor) and SB-431542 (TGF-β1 receptor kinase inhibitor). Fibroblast differentiation was investigated by means of gene expression of αSMA using RT-qPCR, it was shown to be well correlated with the protein level of αSMA (FIGS. 5B and 5C) and as published elsewhere. As shown in FIG. 7A, αSMA gene expression was significantly reduced when treated with blebbistatin, Y-27632 and SB-431542 but not with verteporfin. As has been reported, removing TGF-β1 or reducing TGF-β1 receptor activity could trigger apoptosis in myofibroblast. To address this, a live/dead assay was performed, and the results were quantified via flow cytometry to check the viability of cells treated with different inhibitors. As shown in FIG. 7B, only cells treated with SB-431542 demonstrated a significant increase in cell death, which is in line with other reports, as TGF-β1 is essential in maintaining myofibroblasts. This result also suggests that the reduction in αSMA by treatment with SB-431542 could be caused by cell death, as previously reported. On the other hand, blebbistatin and Y-27632 reduced αSMA gene expression without harming cells, suggesting that cell contractility might be associated with fibroblast differentiation in aligned matrices. In general, cell contractility is upregulated when the small GTPase RhoA activates its downstream effector ROCK. ROCK further upregulates phosphorylation of the myosin light chain. Therefore, inhibiting both myosin and ROCK downregulates cell contractility and can result in diffuse integrin distribution and a lack of focal adhesion in cells. Interestingly, treatment with both cell contractility inhibitors did not affect cell orientation in the direction of matrix alignment (FIG. 7C), only αSMA expression. In line with these findings, cell contractility has been demonstrated to mechanically trigger fibroblast differentiation. In addition, loss of myosin II has been found to inhibit fibroblast differentiation. Furthermore, pharmacological targeting of mechanical contraction in myofibroblasts led to a reduction in the progression of fibrosis in a mouse model. Additionally, other selective ROCK inhibitors could also reduce fibroblast differentiation and matrix remodeling in vitro. All these reports supported our finding that cell contractility might be a key player in fibroblast differentiation and could be a potential regulator in the mechanosensing of fibroblasts in response to matrix alignment.

In summary, it was found that inhibiting cell contractility-associated pathways, both through myosin or ROCK, can reduce fibroblast differentiation in aligned collagen, whereas inhibition of YAP signaling minimally affects differentiation. Inhibition of TGF-β1-associated signaling using SB-431542 led to a reduction in αSMA expression due to cell death. The inhibition of cell contractility did not change the directional cell orientation in the direction of matrix alignment. A schematic illustration of the proposed mechanism of how matrix alignment triggered fibroblast differentiation is depicted in FIG. 7D.

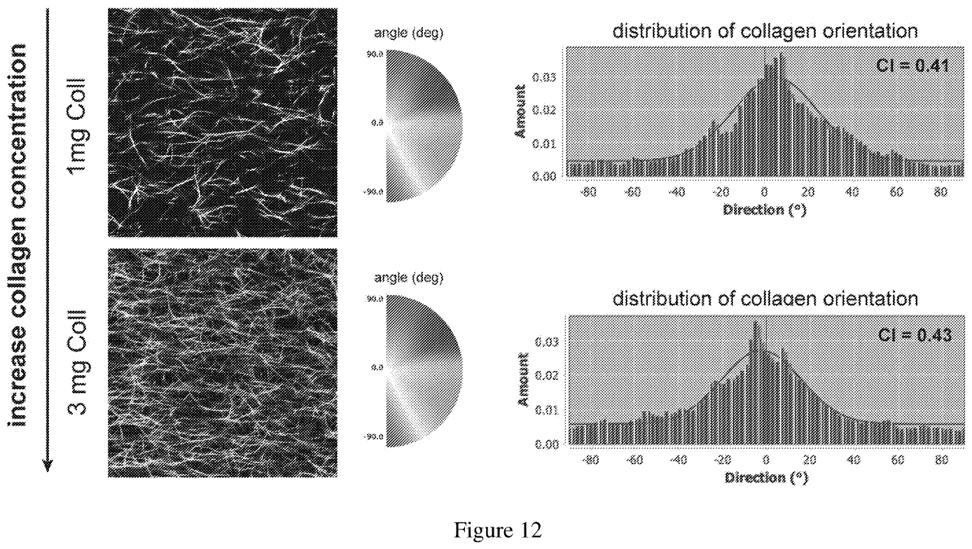

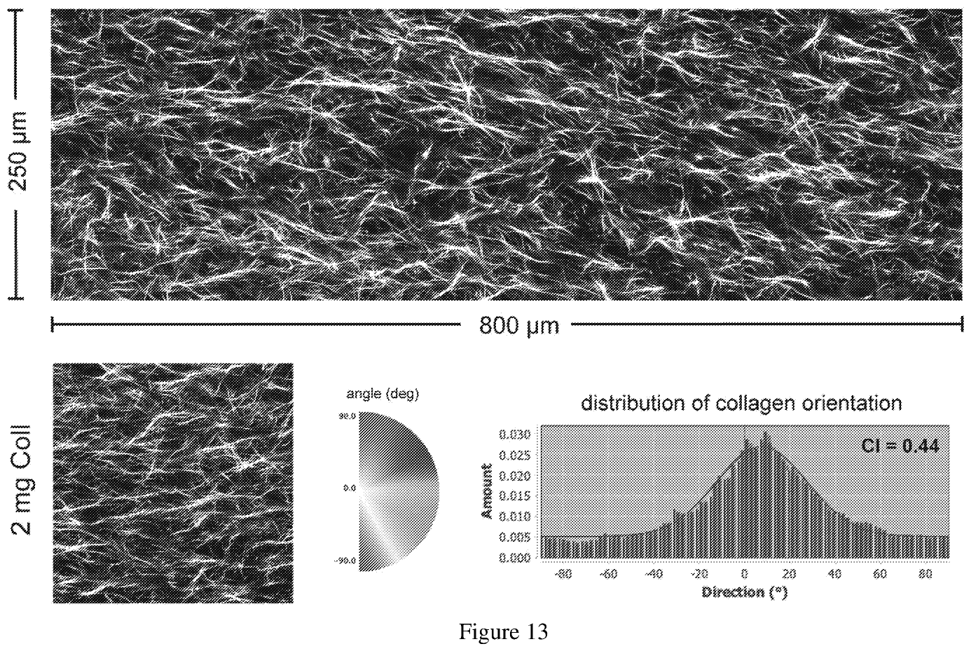

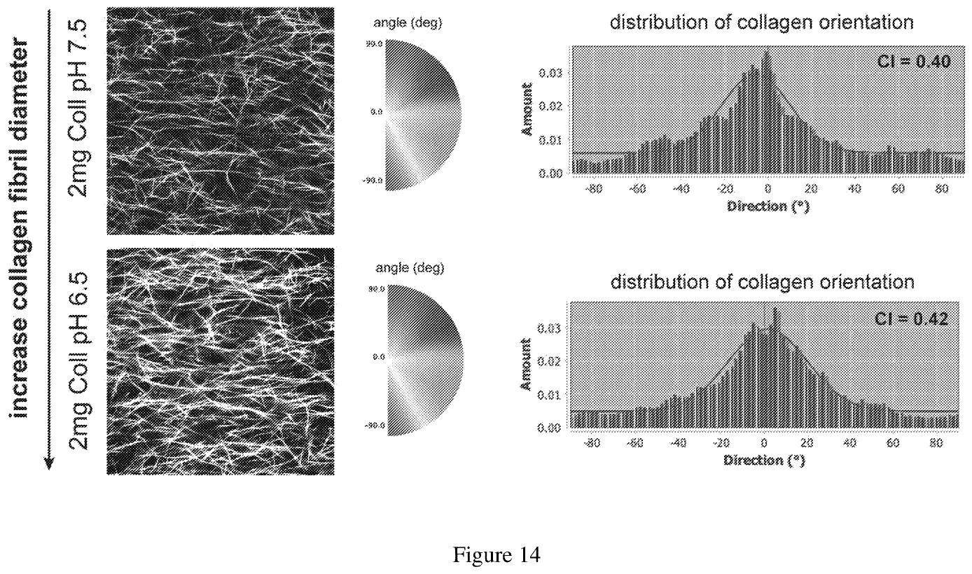

General discussion and conclusion. Collagen fibril alignment represents a key characteristic of fibrotic tissue. Recapitulating such microenvironments biomimetically in vitro will pave the way for understanding molecular mechanisms, better disease modeling and finding novel therapeutic approaches for different pathologies. It has been shown that in vivo fibrosis models have been limited due to species differences between animal and human fibrosis mechanisms, which complicate the interpretation of findings for translation purposes. In addition, the complexity and heterogeneity of the tissue, including biophysical features and compositions, are distinct between different disease stages and can instruct different cell functions. Establishing a well-defined model in which both biophysical and biochemical parameters can be fine-tuned will provide an in-depth understanding of how specific cues regulate distinct cell functions. As exemplified in this work, we established a simple, robust, and noninvasive approach to fine-tune the fibril alignment degree of 3D collagen matrices. In addition, this approach allows for the reconstitution of 3D aligned matrices regardless of the concentration of collagen monomers (as shown in FIG. 12) and their source (as demonstrated in FIG. 13), while also providing the ability to modify fibril characteristics (as illustrated in FIG. 14). It also supports the 3R principle (replacement, reduction, and refinement) of animal usage in research. Using the model, it could be shown that collagen fibril alignment can mechanically trigger fibroblast differentiation, which has been confirmed using functional transcriptome analysis and immunostaining of myofibroblast markers, as well as assessment of matrix remodeling capabilities. Fibroblast differentiation could not be observed in crosslinked random matrices, which exhibit higher bulk matrix stiffness than aligned matrices. Previous reports have indicated that the crosslinking of collagen using EDC could have an impact on the binding sites of integrins on cells, and in turn, affecting cellular contractility and the differentiation of fibroblasts. In our studies, with or without EDC crosslinking, based on αSMA expression and cell proliferation, there was no differences in cellular functions of fibroblasts, but less matrix remodeling which reflect reduced cell contractility. This finding suggests that the alignment of collagen fibrils, rather than the overall stiffness of the matrix, triggers fibroblast differentiation. However, it remains unclear whether an increase in collagen mechanics within aligned collagen matrices would also facilitate the progression of fibroblast behavior. These data also manifest that fibroblast differentiation in aligned matrices appears to be cell contractility dependent, which has been shown using inhibition of myosin II and ROCK. However, blocking cell contractility does not affect the cell orientation of fibroblasts, which might lead to fibroblast differentiation again after removing the inhibitor. In addition, it was demonstrated that matrix organization and stiffness can trigger distinct expression of cytokines and matrix components in fibroblasts, creating specific cellular microenvironments. In summary, there is a contractility-dependent fibrotic response to aligned collagen as seen through hallmark αSMA protein expression, characteristic cell function depicted by matrix remodeling, and transcriptomic analyses. This highlights the importance of physical cues in the regulation of cell functions, especially in the fibrosis and cancer microenvironment, where collagen alignment is mainly presented. It is important to note that collagen fibril alignment is not the only biophysical cue present in fibrotic tissues. Studies have shown that an increase in collagen fibril thickness can also trigger fibroblast differentiation, also via contractility. This highlights the importance of fibroblast cell contractility as a key mechanical cue in fibrotic formation and suggests that its associated pathways could be targeted for therapeutic interventions. However, it is still unclear to what extent collagen fibril thickness and alignment jointly modulate this process. Overall, the 3D aligned collagen matrices can be further extended for the in vitro engineering of cardiac and skeletal tissues, which can be used as a platform for disease modeling of these respective tissue systems, alongside fibrosis and cancer microenvironment studies.

### EXPERIMENTAL SECTION

Reconstitution of collagen matrices with and without fibril alignment: Collagen solution was prepared according to methods known in the art. Briefly, rat tail type I collagen (Advanced Biomatrix, Carlsbad, USA) was mixed with 0.1% acetic acid (Sigma-Aldrich, Germany) and 250 mM phosphate buffer at pH 7.5 (Sigma-Aldrich, Darmstadt, Germany) to achieve a collagen concentration of 2 mg/mL.

For reconstitution of collagen alignment using inclined surfaces, a prepared collagen solution was placed onto a glutaraldehyde-coated coverslip (13 mm in diameter; VWR, Darmstadt, Germany). Thereafter, the coverslip was transferred onto a planar surface (0°) or 3D printed inclined surfaces with angles of 7.5°, 15°, 22.5° and 30°. The design of 3D printed inclined surfaces is illustrated in FIG. 1A. Collagen fibrillogenesis was initiated at 37° C., 5% CO₂ and 95% humidity. Reconstituted 3D collagen matrices were washed 3 times with phosphate buffer saline (PBS; Sigma-Aldrich, Darmstadt, Germany) and kept in PBS prior to performing further experiments.

For crosslinked matrices, reconstituted 3D matrices were treated with 20 mM 1-ethyl-3-(3-dimethyl aminopropyl)-carbodiimide (EDC; Sigma-Aldrich, Darmstadt, Germany) prepared in 2-(N-morpholino) ethanesulfonic acid buffer (MES buffer, Sigma-Aldrich, Darmstadt, Germany) at 0.1 M and pH 5, as previously published. Matrices were incubated with crosslinking solution for 2 h at room temperature. Subsequently after chemical crosslinking, matrices were rinsed 5× with PBS (Sigma-Aldrich, Darmstadt, Germany) and equilibrated at these neutral conditions.

Characterization of topological and mechanical properties of 3D collagen matrices: Cell-free 3D collagen matrices were analyzed to assess their topological and mechanical properties, as previously reported. Briefly, for topological analysis, collagen matrices were stained with 50 μM 5-(and-6)-carboxytetramethylrhodamine succinimidyl ester (TAMRA-SE, Sigma-Aldrich, Darmstadt, Germany) for 2 h at room temperature, as previously published. Matrices were visualized using a confocal laser scanning microscope (SP8; Leica, Wetzlar, Germany) with a 40× objective (NA 1.3). Acquired images were 1024×1024 pixels in resolution (xyz-voxel size: 0.13×0.13×5 μm) and a vertical stack size of 21 images (the total z-stack is equivalent to 100 μm). The cLSM stacked images were analyzed in terms of mean pore and fibril diameter using a home-built MATLAB script (MATLAB 2022aa; MathWorks, Natick, USA), as described elsewhere. The distribution of fibril orientation and the coherence index (CI) index were quantified using obtained images using the OrientationJ plug-in of ImageJ (NIH, Bethesda, USA). The CI is in the range between 0 and 1, where 0 corresponds to randomly oriented fibrils, and 1 corresponds to perfect alignment. The coherency was proven for unbiased characterization of fibril alignment when compared to other methods. The quantification was performed at 4 randomly selected positions of each sample from 4 independent samples.

The mechanical properties of cell-free reconstituted collagen matrices were analyzed nondestructively via rheological measurement using ElastoSens Bio 2 (Rheolution, Montreal, Canada), as previously published. Briefly, prepared collagen solution was placed into the holder (Rheolution, Montreal, Canada) and polymerized onto a planar surface (0°) or 3D printed inclined surfaces with angles of 15°, and 30°. For EDC crosslinked matrices, crosslinking solution were added to the reconstituted matrices, as described above in the collagen reconstitution section. Prior assessment of mechanical properties, all matrices were washed 5× with PBS (Sigma-Aldrich, Darmstadt, Germany) and kept in a hydrated condition. For mechanical characterization of cell culture samples, matrices were decellularized by osmotic shock through incubation with distilled water for 1 h prior measurement. The mechanical characterization was performed at least in triplicate.

Cell culture: Primary human dermal fibroblasts (ATCC, Manassas, USA) were maintained in Dulbecco's Modified Eagle Medium (DMEM) supplemented with 10% fetal bovine serum and 1% penicillin-streptomycin. All cell culture materials were purchased from Thermo Fisher Scientific, Dreieich, Germany.

For cell culture, 3×10⁴ fibroblasts were seeded onto reconstituted 3D collagen matrices. Cells were cultured for 3 days under standard cell culture conditions (37° C. with 5% CO₂ and 95% humidity). For differentiation of fibroblasts into myofibroblasts as a control, cells were cultured in DMEM supplemented with 10 ng/mL TGF-β1 (Biolegend, San Diego, USA) for 3 days under standard cell culture conditions.

Imaging-based quantification of cell orientation: Cells were fixed with 4% paraformaldehyde (Biolegend, San Diego, USA) for 10 min at room temperature, followed by permeabilization using 0.1% Triton X100 (Merck KGaA, Darmstadt, Germany) for 10 min at room temperature. Afterwards, cells were stained 24 h with Hoechst-33420 for the cell nucleus (dilution 1:10000 in PBS; Thermo Fisher Scientific, Dreieich, Germany) and phalloidin conjugated with Alexa Fluor 488 for the actin cytoskeleton (dilution 1:250 in PBS; Thermo Fisher Scientific, Dreieich, Germany). Images were gathered by epifluorescence microscopy (Leica, Wetzlar, Germany) using a 20× LD objective (NA 1.3; Leica, Wetzlar, Germany). Cell orientation was analyzed using the OrientationJ plug-in of ImageJ (NIH, Bethesda, USA). The cell orientation index is in the range between 0 and 1, where 0 corresponds to randomly oriented cells, and 1 corresponds to a perfectly oriented cell. The quantification was performed at 4 randomly selected positions of each sample from 4 independent samples.

RNA sequencing and analysis: For RNA isolation, TRIzol (Invitrogen, California, US) was used to extract the total RNA, followed by a purification step using the RNeasy mini kit (Qiagen, Hilden, Germany) as described by the manufacturer's protocol. RNA quantity and quality were quantified using a Nanodrop (Thermo Fisher Scientific, Dreieich, Germany) and a Qi R-NA kit (Thermo Fisher Scientific, Dreieich, Germany). Samples were prepared with an NEB Ultra 11 RNA kit (New England Biolabs, Ipswich, MA, USA) according to the protocol instructions using the NEBNext Poly(A) mRNA Magnetic Isolation module (New England Biolabs, Ipswich, MA, USA) and uniquely dual indexed. The resulting library concentration, size distribution, and quality were assessed on a Qubit 4 fluorometer (Thermo Fisher Scientific, Inc., Dreieich, Germany) with a dsDNA high sensitivity kit (Invitrogen, Carlsbad, CA, USA) and on a 4200 TapeStation using a High Sensitivity D5000 kit (Agilent, Santa Clara, CA, USA). Based on these results, libraries were normalized according to their molarity and pooled and then quantified with a library quantification kit for Illumina platforms (Roche, Basel, Switzerland) on a StepOnePlus qPCR machine (Thermo Fisher Scientific, Dreieich, Germany). Finally, pooled libraries were loaded at 350 μM with 1% PhiX on S2 FlowCell and paired end sequenced (2×150 bp) on a NovaSeq 6000 next generation sequencer (Illumina, San Diego, USA). RNA-Seq was performed in triplicate. The RNA-Seq data processing procedure is provided herein.

RNA-Seq data were merged using the NASQAR toolbox (publicly accessible at http://nasqar.abudhabi.nyu.edu/), and the analysis was performed using iDEP 1.0 (http://bioinformatics.sdstate.edu/idepg/; publicly accessible by South Dakota State University) (10.1186/s1285-018-2486-6). For the analysis of differentially expressed genes (DEGs), DEGs were analyzed with FDR cutoff≤0.05 and FC≥2.0 using DESeq2. Biological pathways were predicted using generally applicable gene-set enrichment for pathway analysis (GAGE).

Quantitative gene expression analysis: Gene expression analysis for fibroblast differentiation was performed using an established protocol. Briefly, TRIzol (Thermo Fisher Scientific, Dreieich, Germany) was used to extract total RNA. The obtained RNA was converted into complementary DNA (cDNA) using a high-capacity cDNA reverse transcription kit (Applied Biosystems, Waltham, USA). The concentration and the ratio of absorbance at 260 nm and 280 nm of cDNA were quantified using a Nanodrop (Thermo Fisher Scientific, Dreieich, Germany) prior to performing gene expression analysis. Ribosomal protein S26 (RPS26) was used as a reference gene. The primers were synthesized by Bioneer (Daejeon, Republic of Korea). The primer sequences are listed in Table 1. qPCR was performed using SYBR Green PCR Master Mix (Applied Biosystems, Waltham, USA). The qPCR procedure was set as follows: denaturation for 5 min at 95° C.; 45 cycles of denaturation (95° C., 15 s), annealing under primer-specific conditions (30 s) and target gene-specific extension (30 s at 72° C.). The fluorescence signal was measured for 20 s at 72° C. To confirm the specificity of the PCR products, melting curve analysis was performed at the end of each run. Experiments were performed in 4 replicates.

αSMA staining and image analysis: Cells were briefly fixed with 4% paraformaldehyde (Biolegend, San Diego, USA) for 10 min at room temperature, followed by permeabilization using 0.1% Triton X100 (Merck KGaA, Darmstadt, Germany) for 10 min at room temperature. Afterwards, cells were stained for 24 h with Hoechst-33420 for the cell nucleus (dilution 1:10000 in PBS; Thermo Fisher Scientific, Dreieich, Germany) and phalloidin conjugated with Alexa Fluor 549 for the actin cytoskeleton (dilution 1:250 in PBS; Thermo Fisher Scientific, Dreieich, Germany). For additional staining of alpha-smooth muscle actin (αSMA), cells were blocked with 1% bovine serum albumin for 1 h at room temperature, incubated with mouse anti-human αSMA (dilution 1:250 in PBS; Biolegend, San Diego, USA) overnight at 4° C., and incubated with goat anti-mouse IgG conjugated with Alexa Fluor-488 (dilution 1:250 in PBS; Thermo Fisher Scientific, Dreieich, Germany) for 2 h. Cells were washed three times with PBS after each step. Cell imaging was performed using an epi-fluorescence microscope (DMi8 S; Leica. Wetzlar, Germany) using a 20× LD objective (NA 1.3; Leica, Wetzlar, Germany). The percentage of αSMA-positive cells was determined by manual counting of cells positive for αSMA. The quantification was performed at 4 randomly selected positions of each sample from 4 independent samples.

Quantitative analysis of cytokine secretion: To analyze cytokines secreted by cells, cell culture supernatants were collected after 3 days of culture. Bead-based multiplex immunoassay for CCL2, CXCL8, IL6, TGF-β1 and VEGF (Biolegend, San Diego, USA) was utilized to quantify cytokines following instructions by the manufacturer. Samples were analyzed using an Attune NxT Flow Cytometer equipped with an autosampler (Thermo Fisher Scientific, Dreieich, Germany). Data analysis was performed by applying a five-parameter curve fitting algorithm using LEGENDplex™ data analysis software (Biolegend, San Diego, USA). Experiments were performed in 4 replicates.

Treatment with inhibitors: Fibroblasts cultured onto matrix reconstituted on inclined surfaces with an angle of 30° were treated with 1 μM verteporfin (YAP inhibitor; Sigma-Aldrich, Darmstadt, Germany), 10 μM blebbistatin (myosin inhibitor; Tocris, Bristol, United Kingdom), 20 μM Y-27632 (Rho-associated kinase (ROCK) inhibitor; Tocris, Bristol, United Kingdom) and 2 μM SB-431542 (TGF-31 receptor kinase inhibitor; Sigma-Aldrich, Darmstadt, Germany) for 3 days. After 3 days of treatment, RNA was extracted for the study of αSMA expression using qPCR, as stated in the quantitative gene analysis section. Experiments were performed in 6 replicates.

Quantitative analysis of cell number and dead cells: Collagen matrices were digested using 2 mg/mL type IV collagenase (Thermo Fisher Scientific, Dreieich, Germany) for 15 min under standard cell culture conditions. Afterwards, cells were stained with DRAQ7 (dead cell staining dye; Biolegend, San Diego, USA) for 10 min on ice. Cells were analyzed using an Attune NxT Flow Cytometer equipped with an autosampler (Thermo Fisher Scientific, Dreieich, Germany). To analyze cell proliferation, the cell count obtained from the flow cytometer was normalized to the number of cells cultured on a random matrix (0°). The percentage of dead cells was quantified by counting the number of cells that were positive for DRAQ7. At least 20,000 cells were analyzed per condition and replicate. Experiments were performed in 6 independent replicates.

Statistical analysis: Experiments were performed in at least four replicates unless otherwise stated. Error bars indicate standard deviation (SD). Levels of statistical significance were determined by a Mann-Whitney test using GraphPad Prism 9 (GraphPad Software, San Diego, USA). The significance level was set at p<0.05.

Processing of RNA Sequencing data. Raw FASTQ sequenced reads were first assessed for quality using FastQC v0.11.5 (available online at www.bioinformatics.babraham.ac.uk/pro ects/fastqc/). The reads were then passed through Trimmomatic v036 for quality trimming and adapter sequence removal with the following parameters (ILLUMINACLIP: trimmomatic_adapter.fa:2:30:10 TRAILING:3 LEADING:3 SLIDINGWINDOW:4:15 MINLEN:36). The surviving trimmed read pairs were then processed with Fastp to remove poly-G tails and Novaseq/Nextseq-specific artifacts. Following quality trimming, the reads were assessed again using FastQC. After QC and QT, the reads were aligned to the human reference genome GRCh38.p4 using HISAT2 with the default parameters and by providing the -dta flag. The resulting SAM alignments were then converted to BAM format and coordinate sorted using SAMtools v1.3.1. The sorted alignment files were then passed through HTSeq-count v0.6.1p1 using the following options (-s no -t exon -I gene_id) for raw count generation. Concurrently, the sorted alignments were processed through Stringtie v1.3.0 for transcriptome quantification. Briefly, the process was stringtie->stringtie merge (to create a merged transcriptome GTF file of all the samples)->stringtie (this time using the GTF generated by the previous mnerging step). Finally, Qualimap v2.2.2 was used to generate RNA-Seq-specific QC metrics per sample.

TABLE 1
Primer list

| | | | SEQ | |
|---|---|---|---|---|
| | | | ID | Accession |
| Gene | Primer | Sequence (5′→3′) | NO: | number |
| RPS26 | forward | CAATGGTCGTGCCAAAAAG | 1 | NM_ |
| | reverse | TTCACATACAGCTTGGGAAGC | 2 | 001029 |
| αSMA | forward | AGACCCTGTTCCAGCCATC | 3 | NM_ |
| (ACTA2) | reverse | TGCTAGGGCCGTGATCTC | 4 | 001141945.1 |

TABLE 2
Comparison of various techniques for reconstituting
3D collagen matrices with fibril alignment.

| | | Adjustable | | |
|---|---|---|---|---|
| | Ease of | alignment | Repro- | |
| Method | fabrication | degree | ducibility | Throughput |
| Microfluidic devices | ++ | ++ | ++ | + |
| Paramagnetic | ++ | + | + | + |
| microbeads | | | | |
| Cyclic mechanical | ++ | + | ++ | + |
| stretching | | | | |
| Constrained cellular | + | + | + | + |
| compaction | | | | |
| Microextrusion 3D | ++ | + | +++ | +++ |
| printing | | | | |
| Inclined surface | +++ | +++ | +++ | +++ |
| (this work) | | | | |

### Example 2

This example provides a description the methods of the present disclosure.

A recent study using a mouse model demonstrated that heightened stiffness in cancer tissue leads to the accumulation of M2-like macrophages, in contrast to their more compliant counterparts. However, it remains unclear whether these microenvironments attract macrophages for accumulation or promote macrophage polarization toward M2-like phenotypes. Additionally, cancer tissue is complex, and less is known about which matrix parameters contribute to promoting M2-like macrophages, as matrix stiffening can be caused by various factors, including enhanced crosslinking and collagen fibril alignment. To address this question, 3D collagen matrices with enhanced stiffness achieved through chemical crosslinking, or collagen fibril alignment, were employed. Using these matrix models, THP-1 monocytic cells were differentiated into uncommitted macrophages (M0) and assessed immunophenotyping, cytokine secretion profiling, transcriptome analysis, and inhibition of various signal transduction pathways to elucidate the underlying mechanisms involved in macrophage polarization and mechanosensing. Subsequently, co-culture experiments were performed between macrophages and two types of breast cancer cells, namely MDA-MB-231 and MCF-7, to investigate cell-cell interactions within the reconstructed 3D models.

Establishment of well-defined 3D collagen matrices for cell culture. To recapitulate tissue stiffening, 3D collagen matrices with enhanced stiffness achieved through chemical crosslinking, or collagen fibril alignment, were employed. For chemical crosslinking, reconstituted collagen matrices were post-modified using a zero-length EDC crosslinker, allowing enhancing matrix stiffness while maintaining its microarchitecture. For the reconstruction of aligned matrix, collagen matrices were reconstituted on a 300 inclined surface, as previously described in. This method leverages the sedimentation effects, caused by gravitational forces during collagen fibrillation on the inclined surface, to generate the aligned matrix microarchitecture.

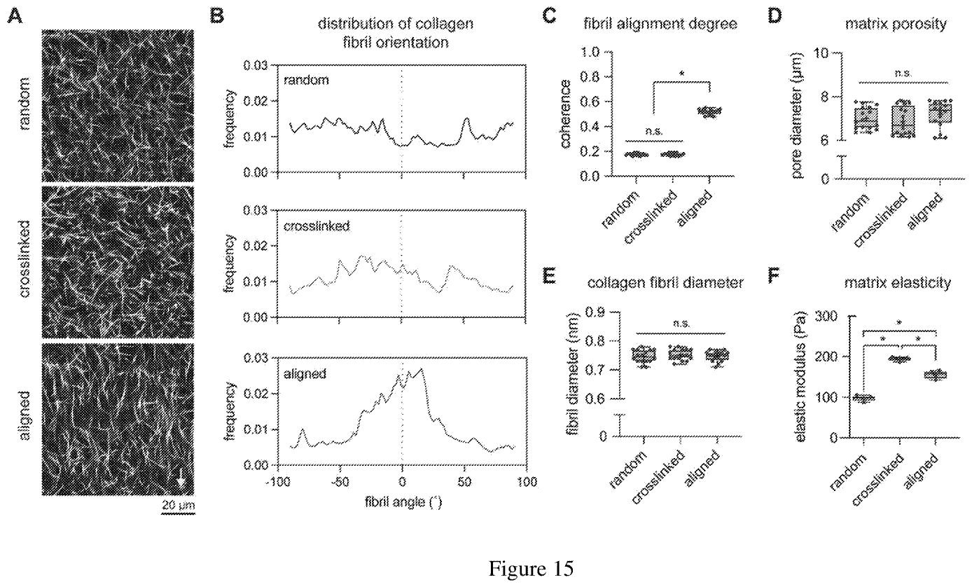

Prior to using the matrices for cell culture, the matrices were visualized and characterized in terms of matrix microarchitecture and elastic elasticity. As shown in FIG. 15A, matrices were fluorescently labeled with TAMRA-SE and visualized using a confocal laser scanning microscope. It is visually apparent that the random and crosslinked matrices exhibit randomly distributed collagen fibrils, while the aligned matrix displays an anisotropic fibril orientation. To quantitatively validate these visual observations, a custom-made image analysis toolbox was employed to analyze the distribution of collagen fibril orientation and the coherence index. As shown in FIG. 15B, random and crosslinked matrices displayed a uniformly distributed fiber orientation, while aligned matrices exhibited a Gaussian distribution. To further examine the fibril orientation, the coherence index, a quantitative measure on a scale from 0 to 1, where 0 represents a random fibril distribution and 1 signifies perfect fibril alignment, was calculated. These data revealed that the coherence index is approximately 0.18 and the value remained consistent for both random and crosslinked matrices (FIG. 15C), suggesting a random matrix. In contrast, a significant increase in the coherence index was observed in the aligned matrices, confirming the successful reconstruction of a matrix with aligned fiber orientation, as visually observed (FIG. 15C). Although the coherent index found is 0.57, this is due to the nature of collagen assembly which cannot compare to the perfect alignment found in printed materials. However, the coherence index of the aligned matrix resembles well with collagen aligned in the human fibrotic conditions. In addition to analyzing fibril alignment, we also quantitatively characterized other topological and mechanical properties of the reconstituted matrices, including matrix porosity, collagen fibril diameter, and elasticity. Our data revealed no significant differences in matrix porosity and collagen fibril diameter among the established matrix conditions (FIGS. 15D and 15E). Quantifying matrix elasticity revealed a significant increase in the elastic modulus of the crosslinked and aligned matrices when compared to the random matrix (FIG. 15F). An increase in the elastic modulus in an aligned matrix compared to a random matrix is well correlated with previous reports.

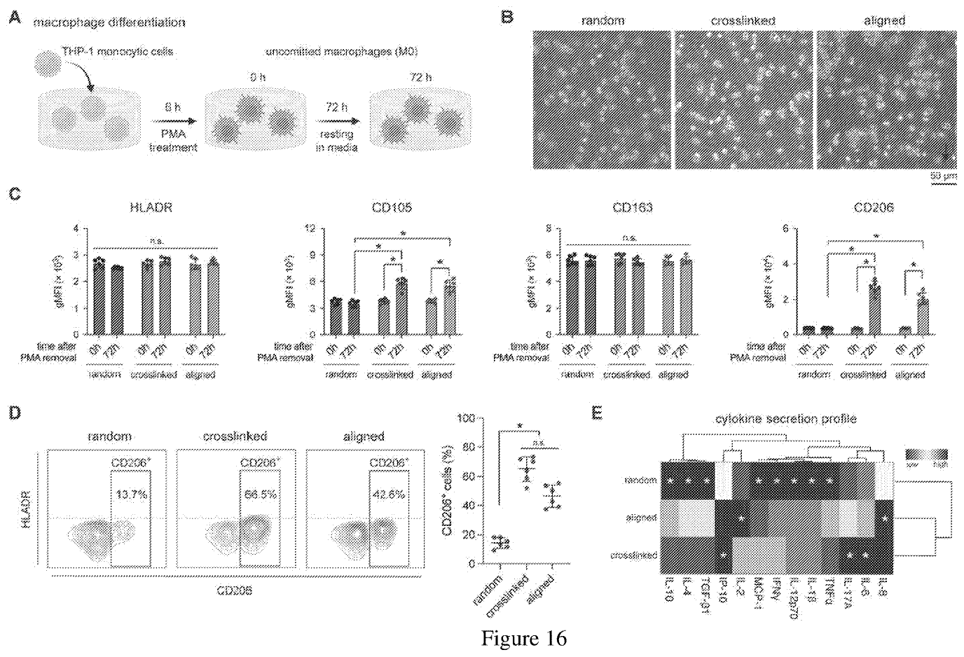

Both stiff matrices induce macrophage polarization towards M2-like phenotype. The reconstituted matrices were utilized to examine the effects of matrix stiffening on macrophage phenotype. In the study of macrophage differentiation within these matrices, THP-1 monocytic cells were employed. These cells were seeded onto the matrices and differentiated into uncommitted macrophages (M0) using phorbol 12-myristate 13-acetate (PMA) for 6 hours, followed by a resting period in cell culture media for 72 hours. This process of macrophage differentiation is illustrated in FIG. 16A.

FIG. 16B presents representative images of macrophages cultured on various matrices for 72 hours, as visualized through bright-field microscopy. These images reveal that macrophages maintain a round morphology across different matrix conditions. To determine if matrix stiffening influences macrophage polarization into specific phenotypes, cell surface markers and cytokine secretion profiles were analyzed. As depicted in FIG. 16C, we measured the expression levels of HLADR, CD105, CD163, and CD206 via flow cytometry. HLADR serves as a key marker for pro-inflammatory (M1) macrophages, while CD105, CD163, and CD206 are markers for anti-inflammatory (M2) macrophages. Notably, M2a macrophages show higher expression of CD105 and CD206, whereas M2c macrophages predominantly express CD163. These results indicated an enhanced expression of CD105 and CD206 in macrophages cultured on crosslinked and aligned matrices for 72 hours, whereas HLADR and CD163 levels remained constant compared to the expression at 0 h across all matrix conditions. The prevalence of CD206 positive cells was also examined, as this macrophage phenotype is enriched in the cancer microenvironment. FIG. 16D shows a significant increase in CD206 positive cells in both crosslinked and aligned matrices relative to random matrices, with a marginally higher count in crosslinked matrices, though not statistically significant.

From this surface marker analysis, it was inferred that matrix stiffening may facilitate a shift in macrophage phenotype from uncommitted (M0) to anti-inflammatory (M2). To corroborate this hypothesis, cytokine secretion was quantified in macrophages under different matrix conditions after 72 hours. FIG. 16E shows distinct cytokine profiles: macrophages on random matrices secreted higher levels of pro-inflammatory cytokines such as MCP-1, IFNγ, IL-12p70, IL-1β, and TNFα. Conversely, macrophages on crosslinked and aligned matrices demonstrated reduced secretion of these cytokines. Moreover, anti-inflammatory cytokines like IL-4, IL-10, and TGF-β1 were more prevalent in macrophages on both stiff matrices, with a slightly higher secretion in crosslinked matrices. A comparison of cytokine secretion between macrophages on crosslinked and aligned matrices revealed lower levels of IL-17, IL-6, and IL-8 in the crosslinked matrices. These cytokine profiles suggest that macrophages on both crosslinked and aligned matrices exhibit a reduced inflammatory response, evidenced by lower secretion of pro-inflammatory cytokines and heightened secretion of anti-inflammatory cytokines. This aligns with the surface marker analysis (FIGS. 16C and 16E) and supports the polarization of uncommitted macrophages (M0) into an anti-inflammatory phenotype (M2).

In summary, these findings demonstrate that matrix stiffening, achieved through crosslinking and alignment, promotes the polarization of macrophages towards an M2 phenotype, specifically M2a based on increased expression in CD105 and CD206. In contrast, random matrices sustain the uncommitted macrophage phenotype. This insight contributes to understanding the accumulation of M2-like macrophages in stiff cancer tissues.

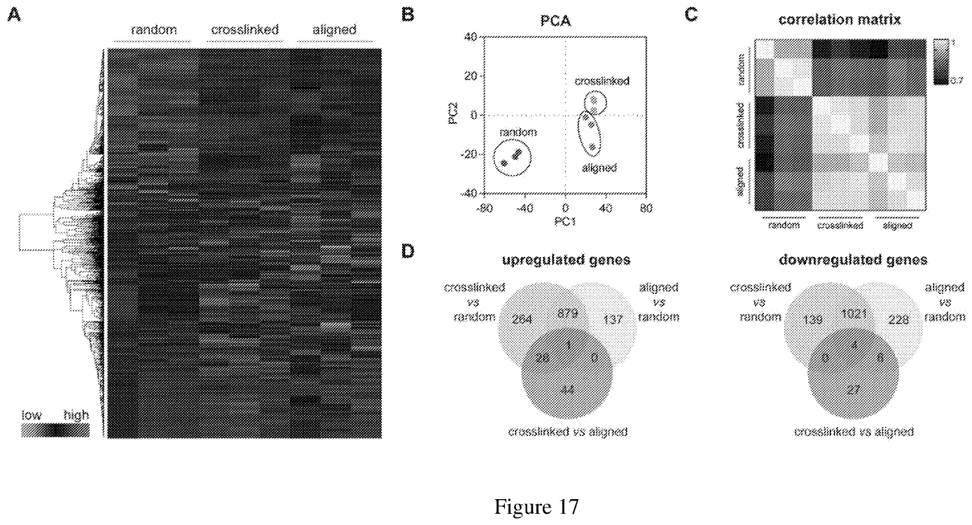

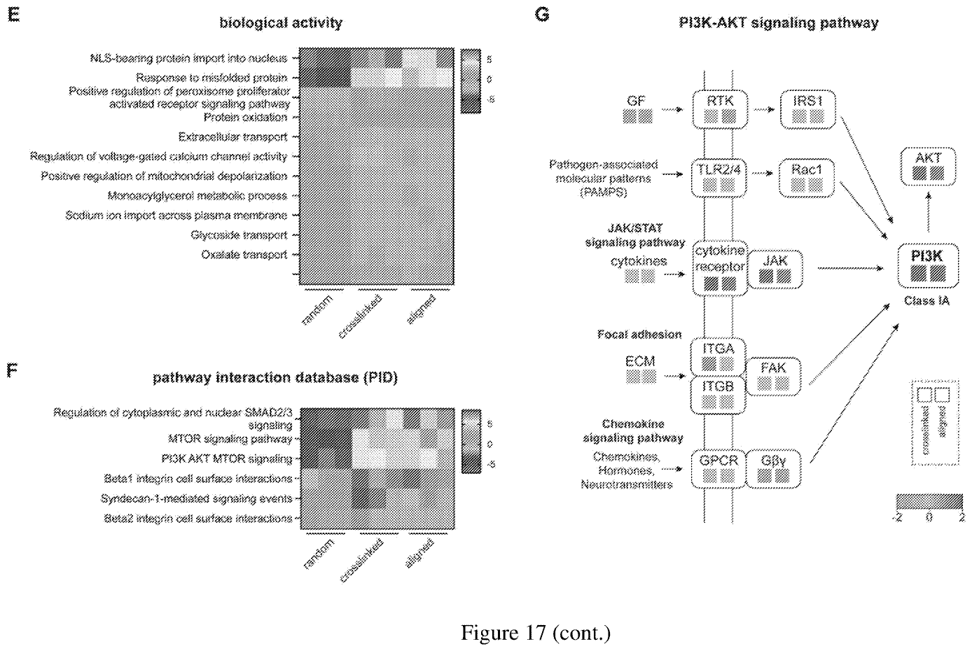

RNA sequencing reveals similarities in transcriptomes and enhancement in gene sets involved in PI3K signaling in both stiff matrices. To comprehend how the matrix influences the polarization of macrophages towards an anti-inflammatory macrophage phenotype, we conducted a functional transcriptome analysis using RNA sequencing. As depicted in FIG. 17A, these findings indicate that the gene expression patterns of macrophages cultured on stiff matrices, whether achieved through crosslinking, or collagen fibril alignment, exhibited similarities. In contrast, macrophages cultured on a compliant random matrix displayed a distinct transcriptome pattern. These results were further substantiated through principal component analysis (PCA) and the correlation matrix, as presented in FIGS. 17B and 17C, respectively.

From the RNA sequencing data, differentially expressed genes (DEGs) were analyzed using a false discovery rate (FDR) threshold of 0.05 and a minimum fold change of 2 for all conditions compared to cells cultured on a random compliant matrix. As illustrated in FIG. 17D, the up- and down-regulated DEGs was visualized in a Venn diagram. While both the crosslinked and aligned conditions exhibited a significant number of DEGs compared to the random conditions, the differences in gene expression changes between them were minimal. To predict enrichment in biological activities and pathway interactions, parametric gene set enrichment analysis (PGSEA) was conducted. This analysis allowed for prediction of biological pathways without bias from the identified DEGs, as DEGs heavily depend on the specific criteria set for the cutoff, in this case, an FDR cutoff of 0.05 and a minimum fold change of 2. As illustrated in FIG. 17E, higher gene set enrichment was observed in biological processes such as NLS-bearing protein import into the nucleus, response to misfolded proteins, positive regulation of the peroxisome proliferator-activated receptor signaling pathway, and protein oxidation in both stiff matrices (crosslinked and aligned samples). Previous reports have indicated that the positive regulation of the peroxisome proliferator-activated receptor signaling pathway promotes macrophage polarization towards anti-inflammatory phenotypes. For the analysis of curated pathway interaction databases (FIG. 17F), it was predicted that the regulation of cytoplasmic and nuclear SMAD2/3, the MTOR signaling pathway, and the PI3K-AKT-MTOR signaling pathway is upregulated in both stiff matrices (crosslinked and aligned samples). Prior studies have shown that PI3K-AKT-MTOR activation is a crucial step in the M2 activation of macrophages in response to IL-4. As PI3K signaling can be activated by various factors, a comprehensive analysis of the potential pathways involved in PI3K activation was conducted. As depicted in FIG. 17G, the JAK-STAT signaling pathway is predicted to be active based on the higher expression of cytokine receptors and gene sets associated with JAK.

Based on these functional transcriptome analyses, it was hypothesized that the activation of the JAK-STAT signaling pathway, as well as PI3K, may be due to the increased secretion of IL-4, as illustrated in FIG. 17E. The activation of the IL-4 receptor signaling pathway involves JAK/STAT-mediated phosphorylation of STAT6, leading to the activation of IRS-2 and downstream activation of PI3K, AKT, and mTOR.

Although the present disclosure has been described with respect to one or more particular embodiments and/or examples, it will be understood that other embodiments and/or examples of the present disclosure may be made without departing from the scope of the present disclosure.

1. A method for aligning protein fibrils comprising:
disposing a disaggregated protein capable of fibrillization on a substrate;
arranging the substrate onto a surface that is oblique to a level plane;
initiating fibrillization of the disaggregated protein;
wherein following fibrillization the protein fibrils have a coherence index (CI) of greater than or equal to 0.15 but less than 1.

2. The method according to claim 1, wherein the disaggregated protein is collagen.

3. The method according to claim 2, wherein the collagen is type I collagen.

4. The method according to claim 1, wherein the substrate is oblique at an angle of 0.1° to 45°, relative to a flat and level surface.

5. The method according to claim 4, wherein the angle is 7.5° to 30°.

6. The method according to claim 1, wherein the CI is 0.15 to 0.5.

7. The method according to claim 1, wherein the initiating fibrillization comprises heating the disaggregated protein at temperature around 37° C. and around 95% humidity and in an atmosphere comprising about 5% CO₂.

8. A hydrogel matrix comprising a plurality of protein fibrils, wherein the protein fibrils are at least partially aligned and have a coherence index (CI) of greater than or equal to 0.15 but less than 1.

9. The hydrogel matrix according to claim 8, wherein the protein fibrils are collagen fibrils.

10. The hydrogel matrix according to claim 9, wherein the collagen fibrils are type I collagen fibrils.

11. The hydrogel matrix according to claim 8, wherein the hydrogel matrix has an average pore diameter of ~6 to 10 μm.

12. The hydrogel matrix according to claim 8, wherein the protein fibrils have an average fibril diameter of 0.65 to 0.725 μm.

13. The hydrogel matrix according to claim 8, wherein the CI is 0.15 to 0.5.

14. The hydrogel matrix according to claim 8, wherein the collagen matrix has an elastic modulus of greater than 105 Pa.

15. A cell culture matrix comprising the hydrogel matrix according to claim 8.

16. A substrate for drug screening comprising the hydrogel matrix according to claim 8.

17. A tissue mimetic comprising the hydrogel matrix according to claim 8.

18. A method for differentiating cells comprising
disposing cells into the cell culture matrix according to claim 15;
optionally, disposing an activator into the cell culture matrix;
culturing the cells in the cell culture matrix; and
optionally, isolating the differentiated cells.

19. An assembly including a first polygonal structure and a second polygonal structure, wherein the first polygonal structure and the second polygonal structure are at least partially separated by a gap, and wherein the first polygonal structure and the second polygonal structure have a base and an oblique surface relative to the base, wherein the assembly is configured to hold a work piece.

20. The assembly according to claim 19, wherein the work piece is a substrate.

21. The assembly according to claim 20, wherein the substrate has a plurality of protein fibrils disposed thereon, wherein the protein fibrils have a coherence index (CI) of greater than or equal to 0.15 but less than 1.
